(19) United States
(12) Patent Application Publication
(10) Pub. No.: US 2025/0381101 A1
(43) Pub. Date: Dec. 18, 2025

(54) HIGHLY FLOWABLE DENTAL CURABLE COMPOSITION

(71) Applicant: SHOFU INC., Kyoto-shi (JP)

(72) Inventors: Kenzo YAMAMOTO, Kyoto-shi (JP); Toko FURUTANI, Kyoto-shi (JP); Naoya KITADA, Kyoto-shi (JP)

(73) Assignee: SHOFU INC., Kyoto-shi (JP)

(21) Appl. No.: 19/233,531

(22) Filed: Jun. 10, 2025

(30) Foreign Application Priority Data
Jun. 13, 2024 (JP) 2024-095933

(51) Int. Cl. A61K 6/76

(52) U.S. Cl. CPC A61K 6/76

(57) ABSTRACT

A dental curable composition, comprising an (A) polymerizable monomer, a (B) polymerization initiator, and a (C) filler,
wherein the (C) component comprises
a (C1) inorganic filler having X-ray radiopacity and
a (C2) hydrophobized silica fine particle having a primary particle size of less than 0.1 μm,
wherein the (C1) component is surface-treated with a hydrophobic silane coupling agent of formula 1, and
a mass ratio of the (C1) component and formula 1 ((C1) component:compound of formula 1) is 100:0.01 to 1,
wherein flowability, which is a distance moved by the dental curable composition with an elapsed time of one minute after 0.05 g of the dental curable composition is placed in a lump on a horizontally placed slide glass plane, and the slide glass is fixed vertically to the horizontal plane, is 10 mm or more.

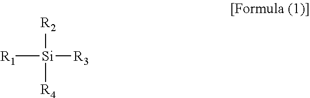

wherein R₁ is an alkyl group having 1 to 11 carbon atoms, which may have one or more selected from a halogen, an aromatic ring, an aliphatic ring, a polymerizable group, —O—, —S—, —NH—, —C(O)—O—, —O—C(O)—, —C(O)—NH—, —O—C(O)—NH—, or —NH—C(O)—O—, and R₂, R₃, and R₄ represent an alkoxy group or an alkyl group having 1 to 4 carbon atoms, or a halogen, and may be the same or different.

There is provided a dental curable composition having high flowability while having X-ray radiopacity, as well as favorable operability when placed in a bottle container, and excellent long-term stability.

### TECHNICAL FIELD

The present invention relates to a highly flowable dental curable composition.

### BACKGROUND ART

Highly flowable dental curable compositions have been used in dental adhesives, dental composite resins, dental core construction materials, dental resin cement, dental coating materials, dental pit and fissure sealants, dental manicure materials, materials for dental 3D printers, materials for orthodontics, and the like.

Patent Literature 1:2006-219439 discloses a dental adhesive composition having X-ray radiopacity, and Patent Literature 2:2022-120710 discloses a two-component type dental adhesive composition.

### CITATION LIST

### Patent Literature

Patent Literature 1: JP 2006-219439 A
Patent Literature 2: JP 2022-120710 A

### SUMMARY

### Technical Problem

However, these compositions have problems with respect to favorable operability and long-term stability when a curable composition having X-ray radiopacity is placed in a bottle container and used.

An object of the present invention is to provide a dental curable composition having high flowability while having X-ray radiopacity, as well as favorable operability when placed in a bottle container and excellent long-term stability.

### Solution to Problem

The present inventors have found as a result of diligent investigations that the above problems can be solved by forming a specific dental curable composition.

The present disclosure provides the following items.

### (Item 1)

A dental curable composition, comprising an (A) polymerizable monomer, a (B) polymerization initiator, and a (C) filler,
wherein the (C) component comprises
a (C1) inorganic filler having X-ray radiopacity and
a (C2) hydrophobized silica fine particle having a primary particle size of less than 0.1 μm,
wherein the (C1) component is surface-treated with a hydrophobic silane coupling agent of formula 1, and
a mass ratio of the (C1) component and formula 1 ((C1) component:compound of formula 1) is 100:0.01 to 1,
wherein flowability, which is a distance moved by the dental curable composition with an elapsed time of one minute after 0.05 g of the dental curable composition is placed in a lump on a horizontally placed slide glass plane, and the slide glass is fixed vertically to the horizontal plane, is 10 mm or more.

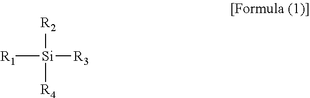

wherein R₁ is an alkyl group having 1 to 11 carbon atoms, which may have one or more selected from a halogen, an aromatic ring, an aliphatic ring, a polymerizable group, —O—, —S—, —NH—, —C(O)—O—, —O—C(O)—, —C(O)—NH—, —O—C(O)—NH—, or —NH—C(O)—O—, and R₂, R³, and R₄ represent an alkoxy group or an alkyl group having 1 to 4 carbon atoms, or a halogen, and may be the same or different.

### (Item 2)

The dental curable composition according to item 1, wherein the (C1) inorganic filler having X-ray radiopacity is aluminosilicate glass comprising one or more elements selected from strontium, barium, lanthanum, zirconium, ytterbium, or gadolinium.

### (Item 3)

The dental curable composition according to item 1, wherein the (C1) inorganic filler having X-ray radiopacity is surface-treated with an acidic compound and a silane coupling agent.

### (Item 4)

The dental curable composition according to item 2, wherein the (C1) inorganic filler having X-ray radiopacity is surface-treated with an acidic compound and a silane coupling agent.

### (Item 5)

The dental curable composition according to item 1, wherein the amount of the (C1) inorganic filler having X-ray radiopacity surface-treated with a silane coupling agent is 0.01 to 0.50 parts by mass relative to 100 parts by mass of the (C1) inorganic filler having X-ray radiopacity.

### (Item 6)

The dental curable composition according to item 2, wherein the amount of the (C1) inorganic filler having X-ray radiopacity surface-treated with a silane coupling agent is 0.01 to 0.50 parts by mass relative to 100 parts by mass of the (C1) inorganic filler having X-ray radiopacity.

### (Item 7)

A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of an (A1) polymerizable monomer having an acidic group,
0 to 50 parts by mass of an (A2) polymerizable monomer having no acidic group,
5 to 50 parts by mass of (D) water, and
0 to 50 parts by of an (E) volatile organic solvent, and
wherein the dental adhesive composition consists of the second agent and the first agent,
wherein the second agent is the second agent according to item 1, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity,
and
3 to 20 parts by mass of the (C2) hydrophobized silica fine particle having a primary particle size of less than 0.1 μm.

### (Item 8)

A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to item 2, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity,
and
3 to 20 parts by mass of the (C2) hydrophobized silica fine particle having a primary particle size of less than 0.1 μm.

### (Item 9)

A kit of a two-step type dental adhesive composition comprising a first agent and a second agent which is the dental curable composition according to item 3, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to item 3, comprising, relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity,
and
3 to 20 parts by mass of the (C2) hydrophobized silica fine particle having a primary particle size of less than 0.1 μm.

### (Item 10)

A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to item 4, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity,
and
3 to 20 parts by mass of the (C2) hydrophobized silica fine particle having a primary particle size of less than 0.1 μm.

### (Item 11)

A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to item 5, comprising, relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity,
and
3 to 20 parts by mass of the (C2) hydrophobized silica fine particle having a primary particle size of less than 0.1 μm.

### (Item 12)

A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to item 6, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity,
and
3 to 20 parts by mass of the (C2) hydrophobized silica fine particle having a primary particle size of less than 0.1 μm.

### Advantageous Effects of Invention

According to the present invention, there is provided a dental curable composition having high flowability while having X-ray radiopacity, as well as favorable operability when placed in a bottle container, and excellent long-term stability.

### BRIEF DESCRIPTION OF THE DRAWINGS

FIG. 1 shows a container for glue used in “Evaluation 3: Favorableness in dischargeability” in Example;

FIG. 2 shows a container for glue used in “Evaluation 3: Favorableness in dischargeability” in Example;

FIG. 3 shows a pour spout cap of a container for glue used in “Evaluation 3: Favorableness in dischargeability” in Example; and

FIG. 4 shows a container for glue used in “Evaluation 3: Favorableness in dischargeability” in Example.

### DETAILED DESCRIPTION OF THE INVENTION

A dental curable composition is a material that can be used to restore an anatomical form of a carious area or a missing tooth, or to improve alignment of teeth and an engagement of teeth. There are various dental curable compositions depending on purposes, and it is preferable that the dental curable composition has X-ray radiopacity, particularly when used to restore an anatomical form of a carious area or a missing tooth. In clinical practice, photography using X-rays may be performed to diagnose secondary caries. In this method, a carious area reduces X-ray radiopacity compared to a healthy tooth, making it possible to diagnose the carious site. In a case in which a dental curable composition having X-ray radiopacity is used to restore an anatomical form of a carious area or a missing tooth, even though dental caries occurs in the circumference of the dental curable composition, there is a difference in X-ray radiopacity between the carious area and the dental curable composition, resulting in facilitation of diagnosis.

On the other hand, in a case in which the dental curable composition does not have sufficient X-ray radiopacity when used to restore an anatomical form of a carious area or a missing tooth, the degree of X-ray radiopacity will be the same as that of the carious site, making diagnosis complicated. For this reason, dental curable compositions imparted with X-ray radiopacity have been developed.

As a method for imparting X-ray radiopacity to the dental curable composition, it is known to compound a filler containing an element such as strontium, barium, lanthanum, zirconium, ytterbium, or gadolinium into the dental curable composition. When a filler containing such an element was used, however, the filler had a high specific gravity and increased a difference in specific gravity with a polymerizable monomer, having resulted in a tendency of precipitation. In a case in which the filler was not subjected to hydrophobization in order to reduce the affinity with the polymerizable monomer, on the other hand, the dental curable composition may have been thickened or decreased in its flowability, and therefore a dental curable composition with desired flowability could not be obtained. For these reasons, it has been difficult to achieve both the X-ray radiopacity and property stability of a dental curable composition.

The present inventors have found as a result of investigations that co-presence of a filler and a smaller amount of a silane coupling agent than usual in a composition, and including a hydrophobized silica fine particle as the filler makes the filler less likely to be precipitated, resulting in a dental curable composition with favorable operability and have thus completed the present invention.

### [(A) Polymerizable Monomer]

The (A) polymerizable monomer (also referred to as “(A) component” in the present invention) contained in the dental adhesive composition of the present invention is not limited and can be any known polymerizable monomer for use. The (A) polymerizable monomer includes an (A1) polymerizable monomer having an acidic group and an (A2) polymerizable monomer having no acidic group. In the polymerizable monomer described in the present invention, its polymerizable group is preferably those that exhibit radical polymerizability, and specifically, from the viewpoint of facilitation of radical polymerization, the polymerizable group is preferably a (meth)acrylic group and/or a (meth)acrylamide group. It is to be noted that the “(meth)acrylic” used herein means acrylic and/or “methacrylic, the “(meth)acryloyl” means acryloyl and/or methacryloyl, the “(meth)acrylate” means acrylate and/or methacrylate, and the “(meth)acrylamide” means acrylamide and/or methacrylamide. A polymerizable monomer having a substituent at the α-position of a (meth)acrylic group and/or a (meth)acrylamide group can also be preferably used. In the present description, a silane coupling agent having a polymerizable group and a particle that has been surface-treated with the silane coupling agent having a polymerizable group are not classified as the (A) polymerizable monomers.

### [(A1) Polymerizable Monomer Having an Acidic Group]

The dental adhesive composition of the present invention contains an (A1) polymerizable monomer having an acidic group (also referred to as “(A1) component” in the present invention) in the first agent. The (A1) polymerizable monomer having an acidic group is not limited and can be used as long as it has one or more polymerizable groups and at least one acidic group such as a phosphoric acid group, a pyrophosphoric acid group, a thiophosphoric acid group, a phosphonic acid group, a sulfonic acid group, or a carboxylic acid group. Including the polymerizable monomer having an acidic group enables adhesiveness to be imparted to teeth and prosthetic devices.

Specific examples of the polymerizable monomer having a phosphoric acid group include one or more selected from 2-(meth)acryloyloxyethyl dihydrogen phosphate, 3-(meth)acryloyloxypropyl dihydrogen phosphate, 4-(meth)acryloyloxybutyl dihydrogen phosphate, 5-(meth)acryloyloxypentyl dihydrogen phosphate, 6-(meth)acryloyloxyhexyl dihydrogen phosphate, 7-(meth)acryloyloxyheptyl dihydrogen phosphate, 8-(meth)acryloyloxyoctyl dihydrogen phosphate, 9-(meth)acryloyloxynonyl dihydrogen phosphate, 10-(meth)acryloyloxydecyl dihydrogen phosphate, 11-(meth)acryloyloxyundecyl dihydrogen phosphate, 12-(meth)acryloyloxydodecyl dihydrogen phosphate, 16-(meth)acryloyloxyhexadecyl dihydrogen phosphate, 20-(meth)acryloyloxyicosyl dihydrogen phosphate, bis[2-(meth)acryloyloxyethyl] hydrogen phosphate, bis[4-(meth)acryloyloxybutyl] hydrogen phosphate, bis[6-(meth)acryloyloxyhexyl] hydrogen phosphate, bis[8-(meth)acryloyloxyoctyl] hydrogen phosphate, bis[9-(meth)acryloyloxynonyl] hydrogen phosphate, bis[10-(meth)acryloyloxydecyl] hydrogen phosphate, 1,3-di(meth)acryloyloxypropyl dihydrogen phosphate, 2-(meth)acryloyloxyethylphenyl hydrogen phosphate, 2-(meth)acryloyloxyethyl-2-bromoethyl hydrogen phosphate, or bis[2-[meth]acryloyloxy-(1-hydroxy methyl)ethyl] hydrogen phosphate; acid chlorides, alkali metal salts, or ammonium salts thereof; or (meth)acrylamide compounds in which ester bonds of these compounds have been replaced with amide bonds.

Specific examples of the polymerizable monomer having a pyrophosphoric acid group include one or more selected from bis[2-(meth)acryloyloxyethyl] pyrophosphate, bis[4-(meth)acryloyloxybutyl] pyrophosphate, bis[6-(meth)acryloyloxyhexyl] pyrophosphate, bis[8-(meth)acryloyloxyoctyl] pyrophosphate, or bis[10-(meth)acryloyloxydecyl] pyrophosphate; acid chlorides, alkali metal salts, or ammonium salts thereof; or (meth)acrylamide compounds in which ester bonds of these compounds have been replaced with amide bonds.

Specific examples of the polymerizable monomer having a thiophosphoric acid group include one or more selected from 2-(meth)acryloyloxyethyl dihydrogen thiophosphate, 3-(meth)acryloyloxypropyl dihydrogen thiophosphate, 4-(meth)acryloyloxybutyl dihydrogen thiophosphate, 5-(meth)acryloyloxypentyl dihydrogen thiophosphite, 6-(meth)acryloyloxyhexyl dihydrogen thiophosphate, 7-(meth)acryloyloxyheptyl dihydrogen thiophosphite, 8-(meth)acryloyloxyoctyl dihydrogen thiophosphate, 9-(meth)acryloyloxynonyl dihydrogen thiophosphate, 10-(meth)acryloyloxydecyl dihydrogen thiophosphate, 11-(meth)acryloyloxyundecyl dihydrogen thiophosphate, 12-(meth)acryloyloxydodecyl dihydrogen thiophosphate, 16-(meth)acryloyloxyhexadecyl dihydrogen thiophosphate, or 20-(meth)acryloyloxyicosyl dihydrogen thiophosphate; acid chlorides, alkali metal salts, or ammonium salts thereof; or (meth)acrylamide compounds in which ester bonds of these compounds have been replaced with amide bonds.

Specific examples of the polymerizable monomer having a phosphonic acid group include one or more selected from 2-(meth)acryloyloxyethyl phenyl phosphonate, 5-(meth)acryloyloxypentyl-3-phosphonopropionate, 6-(meth)acryloyloxyhexyl-3-phosphonopropionate, 10-(meth)acryloyloxydecyl-3-phosphonopropionate, 6-(meth)acryloyloxyhexyl-3-phosphonoacetate, 10-(meth)acryloyloxydecyl-3-phosphonoacetate; acid chlorides, alkali metal salts, or ammonium salts thereof; or (meth)acrylamide compounds in which ester bonds of these compounds have been replaced with amide bonds.

Specific examples of the polymerizable monomer having a sulfonic acid group include one or more selected from 2-(meth)acrylamide-2-methylpropanesulfonic acid or 2-sulfoethyl (meth)acrylate.

The polymerizable monomer having a carboxylic acid group is classified into a (meth)acrylic-based compound having one carboxyl group in the molecule, and a (meth)acrylic-based compound having a plurality of carboxyl groups in the molecule. Specific examples of the (meth)acrylic compound having one carboxyl group in the molecule include one or more selected from (meth)acrylic acid, N-(meth)acryloylglycine, N-(meth)acryloylaspartic acid, 0-(meth)acryloyltyrosine, N-(meth)acryloyltyrosine, N-(meth)acryloylphenylalanine, N-(meth)acryloyl-p-aminobenzoic acid, N-(meth)acryloyl-o-aminobenzoic acid, p-vinylbenzoic acid, 2-(meth)acryloyloxybenzoic acid, 3-(meth)acryloyloxybenzoic acid, 4-(meth)acryloyloxybenzoic acid, N-(meth)acryloyl-5-aminosalicylic acid, N-(meth)acryloyl-4-aminosalicylic acid, 2-(meth)acryloyloxyethyl hydrogen succinate, 2-(meth)acryloyloxyethyl hydrogen phthalate, or 2-(meth)acryloyloxyethyl hydrogen malate; acid halides thereof; or (meth)acrylamide compounds in which ester bonds of these compounds have been replaced with amide bonds. Specific examples of the (meth)acrylic-based compound having a plurality of carboxyl groups in the molecule include one or more selected from 6-(meth)acryloyloxyhexane-1,1-dicarboxylic acid, 9-(meth)acryloyloxynonane-1,1-dicarboxylic acid, 10-(meth)acryloyloxydecane-1,1-dicarboxylic acid, 11-(meth)acryloyloxyundecane-1,1-dicarboxylic acid, 12-(meth)acryloyloxydodecane-1,1-dicarboxylic acid, 13-(meth)acryloyloxytridecane-1,1-dicarboxylic acid, 4-(meth)acryloyloxyethyl trimellitic acid, 4-(meth)acryloyloxybutyl trimellitic acid, 4-(meth)acryloyloxyhexyl trimellitic acid, 4-(meth)acryloyloxydecyl trimellitic acid, or 2-(meth)acryloyloxyethyl-3′-(meth)acryloyloxy-2′-(3,4-dicarboxybenzoyloxy) propyl succinate; acid anhydrides and acid halides thereof; or (meth)acrylamide compounds in which ester bonds of these compounds have been replaced with amide bonds.

### [(A2) Polymerizable Monomer Having No Acidic Group]

The (A2) polymerizable monomer having no acidic group (also referred to as “(A2) component” in the present invention) is not limited and can be used as long as it is a polymerizable monomer having one or more polymerizable groups and no acidic group. Examples of the (A2) polymerizable monomers having no acidic group include one or more selected from a polymerizable monomer having one radical polymerizable group, a polymerizable monomer having two radical polymerizable groups, or a polymerizable monomer having three or more radical polymerizable groups.

Among the (A2) polymerizable monomers having no acidic group, specific examples of the polymerizable monomer having one radical polymerizable group include one or more selected from 2-hydroxyethyl (meth)acrylate, 3-hydroxypropyl (meth)acrylate, 4-hydroxybutyl (meth)acrylate, 2-hydroxypropyl (meth)acrylate, 2-hydroxybutyl (meth)acrylate, 6-hydroxyhexyl (meth)acrylate, 10-hydroxydecyl (meth)acrylate, propylene glycol mono(meth)acrylate, glycerol mono(meth)acrylate, erythritol mono(meth)acrylate, N-methylol (meth)acrylamide, N-hydroxyethyl(meth)acrylamide, N,N-(dihydroxyethyl) (meth)acrylamide, methyl(meth)acrylate, ethyl(meth)acrylate, propyl(meth)acrylate, isopropyl(meth)acrylate, butyl(meth)acrylate, isobutyl(meth)acrylate, benzyl(meth)acrylate, lauryl(meth)acrylate, 2,3-dibromopropyl(meth)acrylate, 3-(meth)acryloyloxypropyltrimethoxysilane, 11-(meth)acryloyloxyundecyltrimethoxysilane, or (meth)acrylamide.

Among the (A2) polymerizable monomers having no acidic group, specific examples of the polymerizable monomer having two radical polymerizable groups include one or more selected from 2,2-bis((meth)acryloyloxyphenyl) propane, 2,2-bis[4-(3-(meth)acryloyloxy)-2-hydroxypropoxyphenyl]propane (commonly known as “Bis-GMA”), 2,2-bis(4-(meth)acryloyloxyphenyl) propane, 2,2-bis(4-(meth)acryloyloxypolyethoxyphenyl) propane, 2,2-bis(4-(meth)acryloyloxydiethoxyphenyl) propane, 2,2-bis(4-(meth)acryloyloxytetraethoxyphenyl) propane, 2,2-bis(4-(meth)acryloyloxypentaethoxyphenyl) propane, 2,2-bis(4-(meth)acryloyloxydipropoxyphenyl) propane, 2-(4-(meth)acryloyloxydiethoxyphenyl)-2-(4-(meth)acryloyloxydiethoxyphenyl) propane, 2-(4-(meth)acryloyloxydiethoxyphenyl)-2-(4-(meth)acryloyloxyditriethoxyphenyl) propane, 2-(4-(meth)acryloyloxydipropoxyphenyl)-2-(4-(meth)acryloyloxytriethoxyphenyl) propane, 2,2-bis(4-(meth)acryloyloxypropoxyphenyl) propane, 2,2-bis(4-(meth)acryloyloxyisopropoxyphenyl) propane, 1,4-bis(2-(meth)acryloyloxyethyl) pyromellitate, glycerol di(meth)acrylate, 1-(acryloyloxy)-3-(methacryloyloxy)-2-propanol, ethylene glycol di(meth)acrylate, diethylene glycol di(meth)acrylate, triethylene glycol di(meth)acrylate, propylene glycol di(meth)acrylate, butylene glycol di(meth)acrylate, neopentyl glycol di(meth)acrylate, polyethylene glycol di(meth)acrylate, 1,3-butanediol di(meth)acrylate, 1,5-pentanediol di(meth)acrylate, 1,6-hexanediol di(meth)acrylate, 1,10-decanediol di(meth)acrylate, 1,2-bis(3-methacryloyloxy-2-hydroxypropoxy) ethane, 2,2,4-trimethylhexamethylenebis(2-carbamoyloxyethyl)dimethacrylate (commonly known as “UDMA”), or 1,2-bis(3-methacryloyloxy-2-hydroxypropoxy) ethane.

Among the (A2) polymerizable monomers having no acidic group, specific examples of the polymerizable monomer having three radical polymerizable groups include one or more selected from trimethylolpropane tri(meth)acrylate, trimethylolethane tri(meth)acrylate, trimethylolmethane tri(meth)acrylate, pentaerythritol tri(meth)acrylate, pentaerythritol tetra(meth)acrylate, dipentaerythritol penta(meth)acrylate, N,N-(2,2,4-trimethylhexamethylene)bis[2-(aminocarboxy) propane-1,3-diol]tetramethacrylate, or 1,7-diacryloyloxy-2,2,6,6-tetraacryloyloxymethyl-4-oxyheptane.

In addition to these polymerizable monomers, use of oligomer or prepolymer having at least one or more polymerizable groups in each molecule is in no way limited. Also, there is no problem even though such an oligomer or a prepolymer may have a substituent such as a fluoro group in the same molecule. The above-described polymerizable monomers can be used singly or in combination with a plurality thereof.

The dental curable composition of the present invention contains the (A) polymerizable monomer. The dental curable composition of the present invention preferably contains the (A2) polymerizable monomer having no acidic group as the (A) polymerizable monomer singly. When the dental curable composition contains the (A1) polymerizable monomer having an acidic group, it is necessary to select the (C1) inorganic filler having X-ray radiopacity that does not interact with the (A1) polymerizable monomer having an acidic group, and when an inorganic filler that interacts with the polymerizable monomer is selected, operability of the dental curable composition may decrease. Regardless of the type of (C1) inorganic filler having X-ray radiopacity, the preferred amount of the (A1) polymerizable monomer having an acidic group that can be compounded is 0 to 5 parts by mass, more preferably 0 to 1 part by mass, and still more preferably none, relative to 100 parts by mass of the (A) polymerizable monomer contained in the dental curable composition.

### <Polymerization Initiator (B)>

The dental curable composition of the present invention contains a (B) polymerization initiator (also referred to as “(B) component” in the present invention). A polymerization method that can be suitably used in the dental curable composition of the present invention includes photopolymerization, chemical polymerization, or dual polymerization including both. The dental curable composition of the present invention preferably contains a photopolymerization initiator.

### [Photopolymerization Initiator]

When the dental curable composition of the present invention contains no chemical polymerization initiator, it contains a photopolymerization initiator. The photopolymerization initiator is a polymerization initiator capable of initiating polymerization by irradiation of light. Examples of the photopolymerization initiator that can be used in the dental adhesive composition of the present invention include one or more selected from a photosensitizer, a photoacid generator, or a photopolymerization accelerator. Known compounds commonly used can be used therefore without any limitations.

Specific examples of the photosensitizer that can be used in the dental curable composition of the present invention include one or more selected from α-diketones, benzoin alkyl ethers, thioxanthones, benzophenones, acylphosphine oxides, or acylgermanium compounds. Examples of the α-diketones include one or more selected from camphorquinone, camphorquinone carboxylic acid, or camphorquinone sulfonic acid. Examples of the benzoin alkyl ethers include one or more selected from benzoin, benzoin methyl ether, or benzoin ethyl ether. Examples of the thioxanthones include one or more selected from 2-isopropylthioxanthone, 2-methoxythioxanthone, 2-hydroxythioxanthone, 2,4-diethylthioxanthone, or 2,4-diisopropylthioxanthone. Examples of the benzophenones include one or more selected from benzophenone, p-chlorobenzophenone, or p-methoxybenzophenone. Examples of the acylphosphine oxides include one or more selected from diphenyl(2,4,6-trimethylbenzoyl) phosphine oxide, or phenylbis(2,4,6-trimethylbenzoyl) phosphine oxide. Examples of the acylgermanium compounds include one or more selected from bisbenzoyldiethylgermanium or bisbenzoyldimethylgermanium.

Examples of the photoacid generator that can be used in the dental curable composition of the present invention include one or more selected from a triazine compound, an iodonium salt-based compound, a sulfonium salt-based compound, or a sulfonic acid ester compound. Among these, one or more selected from the triazine compound or iodonium salt-based compound are preferred because of their high polymerizability when used in combination with a sensitizer. Specific examples of the preferred iodonium salt-based compound include one or more selected from 4-isopropyl-4′-methyldiphenyliodonium tetrakis(pentafluorophenyl) borate, bis(4-tert-butylphenyl) iodonium tetrakis(pentafluorophenyl) borate, bis(4-tert-butylphenyl) iodonium hexafluorophosphate, or diphenyliodonium-2-carboxylate monohydrate.

The photopolymerization accelerator that can be used in the dental curable composition of the present invention can be an amine compound. Examples of the amine compound include one or more selected from p-dimethylaminobenzoic acid ethyl ester, triethanolamine, triisopropanolamine, tribenzylamine, dibenzylglycine ethyl ester, N,N-dimethylaminoethyl acrylate, N,N-diethylaminoethyl methacrylate, or N,N-diisopropylaminoethyl methacrylate.

A combination of the polymerization initiators that can be suitably used in the dental curable composition of the present invention is a combination of the photosensitizer and the amine compound, more specifically, a combination of an α-diketone compound containing camphorquinone and a dialkylbenzoic acid ester compound containing p-dimethylaminobenzoic acid ethyl ester. In addition thereto, it is preferable therefore to include an amine compound containing a hydroxyl group, such as triethanolamine, triisopropanolamine, N,N-bis(2-hydroxyethyl)-p-toluidine, or N,N-bis(2-hydroxypropyl)-p-toluidine, and to include a photopolymerization accelerator such as one or more selected from N,N-dimethylaminoethyl acrylate or N,N-diethylaminoethyl acrylate.

### [Chemical Polymerization Initiator]

The dental curable composition of the present invention can contain a chemical polymerization initiator. Chemical polymerization is a polymerization method for curing it, without requiring special equipment such as a light irradiator, and the chemical polymerization initiator is a polymerization initiator that can initiate chemical polymerization. Known compounds that are commonly used can be used without any limitations.

As specific examples of a transition metal compound that can be used in the dental curable composition of the present invention, a copper (Cu) compound or a vanadium (V) compound, can be preferably used. As the copper (Cu) compound, one or more selected from copper chloride (monovalent), copper bromide (monovalent), copper chloride (divalent), copper acetate (divalent), copper gluconate (divalent), copper acetylacetonate (divalent), copper methacrylate (divalent), and the like, can be used. As the vanadium compound, one or more selected from vanadium acetylacetonate (trivalent), divanadium tetroxide (tetravalent), vanadyl acetylacetonate (tetravalent), vanadium oxide stearate (tetravalent), vanadyl oxalate (tetravalent), vanadyl sulfate (tetravalent), oxo-bis(1-phenyl-1,3-butanedionate) vanadium (tetravalent), bis(maltolato)oxovanadium (tetravalent), vanadium pentoxide (pentavalent), sodium metavanadate (pentavalent), and the like, can be used.

Specific examples of a thiourea compound that can be used in the dental curable composition of the present invention include one or more selected from dimethylthiourea, diethylthiourea, tetramethylthiourea, (2-pyridyl)thiourea, N-methylthiourea, ethylenethiourea, N-allylthiourea, N-allyl-N′-(2-hydroxyethyl)thiourea, N-benzylthiourea, 1,3-dicyclohexylthiourea, N,N′-diphenylthiourea, 1,3-di(p-tolyl)thiourea, 1-methyl-3-phenylthiourea, N-acetylthiourea, N-benzoylthiourea, diphenylthiourea, or dicyclohexylthiourea. Among these, one or more selected from (2-pyridyl)thiourea, N-acetylthiourea, N-benzoylthiourea, or N-benzylthiourea can be preferably used.

Examples of an organic peroxide that can be used in the dental curable composition of the present invention include one or more selected from diacyl peroxides, peroxy esters, dialkyl peroxides, peroxy ketals, ketone peroxides, peroxy esters, peroxy dicarbonates, or hydroperoxides. Among these, examples thereof include one or more selected from t-butylperoxy-2-ethylhexanoate, t-butylperoxybenzoate, t-amylperoxy-2-ethylhexanoate, t-amylperoxyacetate, t-amylperoxybenzoate, 1,1-di(t-butylperoxy)cyclohexane, 1,1-di(t-amylperoxy)cyclohexane, dibenzoyl peroxide, cumene hydroperoxide, t-butyl hydroperoxide, or 1,1,3,3-tetramethylbutyl hydroperoxide.

Examples of other chemical polymerization initiator include one or more selected from a phosphine compound, a sulfinic acid compound, a borate compound, a barbituric acid derivative, or an ascorbic acid compound. Specific examples of the phosphine compound include one or more phosphine compounds selected from triphenylphosphine or 4-(phenylphosphino)benzoic acid. Specific examples of the sulfinic acid compound include one or more sulfinic acid compounds selected from sodium benzenesulfinate, sodium p-toluenesulfinate, or sodium 2,4,6-triisopropylbenzenesulfinate. Examples of the borate compound include one or more borate compounds selected from a sodium salt, a lithium salt, a potassium salt, or a tetrabutylammonium salt of a tetraarylborate compound. Examples of the barbituric acid derivative include one or more barbituric acid derivatives selected from 5-butylbarbituric acid, 1,3,5-trimethylbarbituric acid, 1-cyclohexyl-5-ethylbarbituric acid, or a sodium salt or a calcium salt of the barbituric acid derivative described above. Specific examples of the ascorbic acid compound include one or more ascorbic acid compounds selected from ascorbic acid, ascorbyl 6-palmitate, or a salt compound of the above-described ascorbic acid compound.

It is absolutely acceptable that the polymerization initiator contained in the dental curable composition of the present invention may be subjected to secondary treatment such as micropulverization, support adsorption, or inclusion in a microcapsule, if necessary. Furthermore, these various types of polymerization initiators can be used singly or in combination of two or more thereof, regardless of polymerization modes or polymerization methods.

The dental curable composition of the present invention preferably contains 0.5 to 5.0 parts by mass of polymerization initiator relative to 100 parts by mass of the (A) polymerizable monomer contained in the dental curable composition. The dental curable composition containing 0.5 parts by mass or more of polymerization initiator tends to be favorably cured, and the composition with an amount of 5.0 parts by mass or less tends to have a favorable color tone.

### <(C) Filler>

The dental curable composition of the present invention contains a (C) filler (also referred to as “(C) component” in the present invention). Examples of the (C) filler include an inorganic filler, an organic filler, an organic-inorganic composite filler, and ion-sustained release glass. The (C) fillers also include a (C1) inorganic filler having X-ray radiopacity and a (C2) hydrophobic silica fine particle having a primary particle size of less than 0.1 μm.

Specific examples of the inorganic filler, which is not particularly limited in terms of its chemical composition, specifically include one or more selected from silicon dioxide, alumina, titania, silica-titania, silica-titania-barium oxide, silica-zirconia, silica-alumina, lanthanum glass, borosilicate glass, soda glass, barium glass, strontium glass, glass ceramic, aluminosilicate glass, barium boroaluminosilicate glass, strontium boroaluminosilicate glass, fluoroaluminosilicate glass, calcium fluoroaluminosilicate glass, strontium fluoroaluminosilicate glass, barium fluoroaluminosilicate glass, or strontium calcium fluoroaluminosilicate glass. In particular, one or more selected from barium fluoroaluminosilicate glass, strontium fluoroaluminosilicate glass, fluoroaluminosilicate glass, and the like, which are used in dental glass ionomer cement, resin-reinforced glass ionomer cement, resin cement, and the like, can also be preferably used. The fluoroaluminosilicate glass referred to herein has a basic backbone of silicon oxide and aluminum oxide and contains an alkali metal for introduction of non-crosslinked oxygen. The fluoroaluminosilicate glass further contains an alkaline earth metal containing strontium as a modifying and coordinating ion, and fluorine. The fluoroaluminosilicate glass is also a composition in which a lanthanoid series element is incorporated into a backbone to impart further X-ray opacity. The lanthanoid series element is also incorporated into the composition as a modifying and coordinating ion depending on a composition range.

Specific examples of the organic filler include one or more polymers selected from polymethyl methacrylate, polyethyl methacrylate, a methyl methacrylate-ethyl methacrylate copolymer, an ethyl methacrylate-butyl methacrylate copolymer, a methyl methacrylate-trimethylolpropane methacrylate copolymer, polyvinyl chloride, polystyrene, chlorinated polyethylene, nylon, polysulfone, polyethersulfone, or polycarbonate.

Specific examples of the organic-inorganic composite filler include a filler, a surface of which has been coated by polymerizing a polymerizable monomer, a filler in which it has been mixed with a polymerizable monomer, and the mixture has been then polymerized and pulverized to an appropriate particle size, a filler in which it has been dispersed in a polymerizable monomer, and the mixture has been then emulsion-polymerized or suspension-polymerized, a filler in which it has been dispersed in a polymerizable monomer and a solvent, and the mixture has been then spray-dried then polymerized, and a filler in which it has been dispersed in a solvent, and the mixture has been then spray-dried and then impregnated with a polymerizable monomer followed by polymerization, but the fillers are in no way limited thereto.

As a specific example of the ion-sustained release glass, any ion-sustained release glass can also be limitlessly used as long as it contains one or more glass backbone-forming elements that form a glass backbone and one or more glass modifying elements that modify the glass backbone. These pieces of ion-sustained release glass can be used singly or in combination with a plurality of pieces of the ion-sustained release glass. Also, in the present invention, a glass amphoteric element that plays the role of both of a glass backbone-forming element or a glass modifying element depending on a glass composition is included in the category of glass backbone-forming elements. Specific examples of the glass backbone-forming element contained in ion-sustained release glass include one or more selected from silica, aluminum, boron, or phosphorus, and these can be used singly or in combination thereof. Specific examples of the glass modifying element also include one or more selected from a halogen group element, an alkali metal group element, or an alkaline earth metal group element. Examples of the halogen group element include one or more selected from fluorine, bromine, or iodine. Examples of the alkali metal group element include one or more selected from sodium or lithium. Examples of the alkaline earth metal group element include one or more selected from calcium or strontium. These elements can be used singly or in combination thereof. Among these, it is preferable to include as the glass backbone-forming element, one or more selected from silica, aluminum, or boron, and to include as the glass modifying element, one or more selected from fluorine, sodium, or strontium. Specific examples thereof include silica glass, fluoroaluminosilicate glass, fluoroborosilicate glass, or fluoroaluminoborosilicate glass, which contains one or more selected from strontium or sodium. Furthermore, from the viewpoint of sustained-releasing a fluorine ion, a strontium ion, a borate ion, and an aluminum ion, more preferred is fluoroaluminoborosilicate glass containing strontium. Examples of more preferred glass composition ranges are as follows: SiO₂: 10 to 40% by mass, Al₂O₃: 10 to 35% by mass, B₂O₃: 2.5 to 30% by mass, SrO: 15 to 50% by mass, F: 2.5 to 20% by mass, and Na₂O: 0 to 15% by mass. This glass composition can be confirmed by using instrumental analysis such as elemental analysis, Raman spectroscopy, and X-ray fluorescence analysis, and is fully acceptable as long as the actual measurement values obtained by any of the analysis methods match these composition ranges.

The (C) filler can be treated with a surface treatment agent, represented by a silane coupling agent. The relevant surface treatment agent and surface treatment method are not particularly limited, and any known method can be limitlessly employed, such as a method for spraying a surface treatment agent while stirring a filler in powder form, a method for dispersing and mixing a filler and a surface treatment agent in a solvent, and a method for supplying a silane coupling agent to be formed into vapor or gas to a filler surface. As the silane coupling agent used in surface treatment of the fillers, methyltrimethoxysilane, methyltriethoxysilane, methyltrichlorosilane, dimethyldichlorosilane, trimethylchlorosilane, vinyltrichlorosilane, vinyltriethoxysilane, vinyltris(2-methoxyethoxy)silane, 3-methacryloyloxypropyltrimethoxysilane, 3-chloropropyltrimethoxysilane, 3-glycidoxypropyltrimethoxysilane, 3-(meth)acryloxypropyltrimethoxysilane, 8-(meth)acryloxyoctyltrimethoxysilane, 11-(meth)acryloxyundecyltrimethoxysilane, hexamethyldisilazane, or the like, is preferred. In addition to the silane coupling agents, the fillers can be surface-treated by using a method using a titanate-based coupling agent and/or an aluminate-based coupling agent.

The shape of the (C) filler is not particularly limited, and a filler of any shape, such as spherical, needle-like, platy, crushed, or scaly, can be used. The primary particle size of the filler is preferably in the range of 0.001 μm to 100 μm and more preferably in the range of 0.001 μm to 10 μm.

### [(C1) Inorganic Filler Having X-Ray Radiopacity]

The dental curable composition of the present invention contains a (C1) inorganic filler having X-ray radiopacity (also referred to as “(C1) component” in the present invention). Including the inorganic filler having X-ray radiopacity in the dental curable composition facilitates distinction of dental curable composition from a carious site, leading to expectation of auxiliary effects on diagnosis of secondary caries and a follow-up observation of application site of the dental curable composition.

The (C1) inorganic filler having X-ray radiopacity contains an element having X-ray shielding ability. Specifically, the inorganic filler having X-ray radiopacity is formed by containing one or more elements selected from chromium, iron, zinc, strontium, yttrium, zirconium, tin, tellurium, barium, lanthanum, gadolinium, ytterbium, tantalum, tungsten, or bismuth. Among these elements, the filler preferably contains one or more elements selected from strontium, barium, lanthanum, zirconium, ytterbium, or gadolinium, and the filler is more preferably aluminosilicate glass containing these elements. In the case of the filler being the aluminosilicate glass, more favorable long-term stability can be expected.

The (C1) inorganic filler having X-ray radiopacity has a different degree of X-ray radiopacity depending on the compound type, content, and a compounding method of an element having X-ray shielding ability, so that the (C1) inorganic filler having X-ray radiopacity can be suitably used in the case of containing an element having X-ray shielding ability. The dental curable composition, on the other hand, needs to have X-ray radiopacity, and as such, a ratio of the degree of X-ray radiopacity of a 1 mm cured product of the dental curable composition to that of a 1 mm aluminum plate is preferably 0.5 times or more, more preferably 0.7 times or more, and still more preferably 1 time or more. The ration of 1 time or more facilitates determination of difference from a carious site. In general, the higher the X-ray radiopacity, the more preferable, and the upper limit is approximately 4 times. In the case of less than 0.5 times, on the other hand, the (C1) inorganic filler is not considered to have X-ray radiopacity.

A method for producing the (C1) inorganic filler having X-ray radiopacity is not particularly limited, and it can be produced by a production method such as a melting method or a sol-gel method. Among them, a production method employing a melting method using a melting furnace is preferable from the viewpoint of facilitation of glass composition design including selection of raw materials. The inorganic filler having X-ray radiopacity used herein has an amorphous structure, but has no problem despite partially including a crystalline structure, and further has no problem in spite of being a mixture of glass having an amorphous structure and glass having a crystalline structure. Whether the glass structure is amorphous or not can be confirmed adopting X-ray diffraction analysis or using analytical equipment such as a transmission electron microscope.

The (C1) inorganic filler having X-ray radiopacity is surface-treated with a hydrophobic silane coupling agent of formula 1. In addition to the surface treatment with the hydrophobic silane coupling agent of formula 1, the (C1) inorganic filler having X-ray radiopacity can also be functionalized by known surface treatment. Specific examples of a surface treatment agent used in the surface treatment include a surfactant, a fatty acid, an organic acid, an inorganic acid, a monomer, a polymer, various coupling agents, or a metal alkoxide compound or a partial condensate thereof. Among these surface treatment agents, it is preferable to perform composite surface treatment using an acidic polymer and a silane compound.

The preferred primary particle size of the (C1) inorganic filler having X-ray radiopacity is 0.5 to 100 μm and more preferably 0.5 to 5 μm. In the case of the surface treatment, the filler may be aggregated. In such a case, it is preferable to measure the particle size in a state where aggregates are disaggregated, but it may be difficult to measure it in the disaggregated state. For this reason, the present invention describes a preferred range of the primary particle size prior to the surface treatment. When the primary particle size of the (C1) component is 0.5 μm or more, the dental curable composition tends to have high flowability and favorable operability thereof, and the component (C1) with the primary particle size of 100 μm or less tends to have favorable storage stability.

The structure of the silane coupling agent of formula 1 is exemplified below.

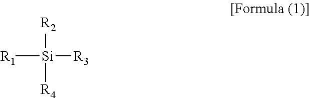

wherein R₁ is an alkyl group having 1 to 11 carbon atoms, which may have one or more selected from a halogen, an aromatic ring, an aliphatic ring, a polymerizable group, —O—, —S—, —NH—, —C(O)—O—, —O—C(O)—, —C(O)—NH—, —O—C(O)—NH—, or —NH—C(O)—O— in the formula, and R₂, R₃, and R₄ are alkoxy groups and alkyl groups having 1 to 4 carbon atoms, or halogens, and may be the same or different.

Specific examples of the alkyl group having 1 to 11 carbon atoms in formula 1 include a linear alkyl group, a branched alkyl group, and a cyclic alkyl group having 1 to 11 carbon atoms.

Specific examples of the alkoxy group or alkyl group having 1 to 4 carbon atoms in formula 1 include a linear alkoxy group, a branched alkoxy group, a cyclic alkoxy group, a linear alkyl group, a branched alkyl group, and a cyclic alkyl group, which have 1 to 4 carbon atoms.

Specific examples of the compound in formula 1 include one or more selected from methylchlorosilane, octadecyltrimethoxysilane, octyltrimethoxysilane, 3-methacryloylpropyltrimethoxysilane, or 8-methacryloyloctyltrimethoxysilane. 3-Methacryloylpropyltrimethoxysilane can be preferably used.

The (C1) inorganic filler having X-ray radiopacity can also be functionalized by known surface treatment in addition to the surface treatment with the silane coupling agent of formula 1 (surface treatment with a hydrophobic surface treatment agent is also called “hydrophobization treatment”). Specific examples of the surface treatment agent used in the surface treatment include a surfactant, a fatty acid, an organic acid, an inorganic acid, a monomer, a polymer, various coupling agents, a metal alkoxide compound and a partial condensate thereof. Among these surface treatment agents, it is preferable to carry out composite surface treatment using an acidic polymer and a silane compound.

The (C1) inorganic filler having X-ray radiopacity is surface-treated with the hydrophobic silane coupling agent of formula 1. A compounding mass ratio of the (C1) component and the compound of formula 1 upon surface treatment ((C1) component (C1):compound of formula 1) is 100:0.01 to 1. It is more preferably 100:0.01 to 0.5. When the compounding mass ratio of the (C1) component to the compound of formula 1 upon surface treatment ((C1) component:compound of formula 1) is 100:0.01 or more, a flowability change is unlikely even after long-term storage, resulting in a dental curable composition having excellent long-term stability. When it is 100:1 or less, it is expected that the dental curable composition will be homogeneous without separation or precipitation even after long-term storage, and that the dental curable composition will have a favorable feeling upon discharge when discharged from a bottle container that has been left stand for a long period of time.

Containers for use, filled with the dental curable composition include a jar container, a syringe container, a bottle container, a pouch container, and the like. Among these, the bottle container has the advantages that its filling process is easier than with a syringe container, that the bottle container tends to have a compact shape for its capacity, and that it facilitates more operability than a jar container. Also, among dental curable compositions that require a small amount to be taken and coated in a thin form, bottle containers are used for a number of dental adhesive materials and dental coating materials. When the dental curable composition is introduced into a syringe container, it can be discharged using pressure from a plunger, but it is difficult for a bottle container to be operated in the same manner. For example, a method is considered in which a bottle container is pressurized by pinching it to discharge the dental curable composition contained therein from a nozzle, however, when the dental curable composition does not have sufficient flowability, it is necessarily pinched tightly, making it difficult to be operated, and in a case in which it is inevitably pinched strongly to discharge the dental curable composition, the amount to be discharged will be ununiform because it depends on the amount of force used by an individual person, as a result of which it may not be possible to discharge an appropriate amount required by the user. For such a reason, the dental curable composition to be used by putting it in a bottle container is preferably a dental composition having high flowability. On the other hand, a material having high flowability, such as the (A) polymerizable monomer used as a matrix, has a low viscosity, so that precipitation of inorganic filler is likely, and in the case of having added a large amount of rheology control agent, the inorganic filler can be inhibited from its precipitation, but may reduce its operability, having made it difficult to achieve both long-term stability and operability. In particular, an inorganic filler having X-ray radiopacity has a higher specific gravity than a silica particle and a polymer particle, so that the inorganic filler has tended to cause precipitation. In the present invention, the present inventors have found that compounding a (C2) hydrophobized silica fine particle having a primary particle size of less than 0.1 μm into the dental curable composition inhibits the (C1) inorganic filler having X-ray radiopacity from being precipitated. In a case in which the (C1) inorganic filler having X-ray radiopacity is not treated with a silane coupling agent, on the other hand, the inorganic filler may have reduced its flowability after long-term storage. In the dental industry, surface treatment is often carried out with more than 1 part by mass of a hydrophobic silane coupling agent relative to 100 parts by mass of inorganic filler, although the amount added depends on the primary particle size. The present inventors have found that the surface treatment with a smaller amount of silane coupling agent than an amount generally used in the dental industry enables the dental curable composition of the present invention to achieve both long-term stability and operability.

The definition of high flowability and a method for evaluating flowability in the present invention will be described below. The method for evaluating flowability is a method for placing 0.05 g of a dental curable composition in a lump on a horizontally placed flat glass slide and next measuring a distance traveled by the dental curable composition 1 minute after the glass slide has been fixed vertically to the horizontal surface. The value of the distance travelled is regarded as the flowability. In this case, the glass slide is not surface-treated and has no convex and concave portions. A material of the glass slide is preferably soda glass. In the present invention, placing in a lump refers to placing the dental curable composition as a lump, and 0.05 g of the dental curable composition must not be divided into two and placed in a thin, spread state. It is preferable to place it in a shape close to a hemisphere. Also, the time from placing 0.05 g of the dental curable composition in a lump on a horizontally placed flat glass slide to fixing it vertically is within 10 seconds and preferably within 5 seconds. The dental curable composition of the present invention has flowability of 10 mm or more, preferably 15 mm or more, and still more preferably 15 mm or more and 35 mm or less. The flowability of 10 mm or more allows the dental curable composition to be appropriately spread to an application site and thereby tends to provide a favorable feeling upon use. The flowability of 35 mm or less makes the filler less likely to be precipitated and tends to render it favorable storage stability.

As a surface treatment method using the silane coupling agent of formula 1 in the (C1) component, known methods such as a wet method, a dry method, and an integral blend method, which are pretreatment methods, can be employed. Among these, the integral blend method is most preferably used.

The pretreatment method refers to a method for subjecting the (C1) component to hydrophobization treatment before a dental curable composition is produced. In the pretreatment method, using a filler which has been hydrophobized so that a compounding ratio of the (C1) component and the compound of formula 1 (component (C1):compound of formula 1) is 100:0.01 to 1 and preferably 100:0.01 to 0.5 in the dental curable composition, allows favorable long-term stability thereof to be expected.

Unlike the wet method and dry method which are pretreatment methods, the integral blend method is a method of surface treatment in the step of mixing the (A) polymerizable monomer and the (C1) component. In other words, the (C1) component is surface-treated upon production of the dental curable composition. In this case, in the mixing step with the (C1) component, the (A) component contained in the dental curable composition may be totally or partially mixed, or the (C1) component may be mixed with a matrix containing the (A) polymerizable monomer and the (B) polymerization initiator. Furthermore, the component (C2) may be simultaneously compounded upon mixing. In the integral blend method, using a filler which has been hydrophobized so that a compounding ratio of the (C1) component and the compound of formula 1 (component (C1):compound of formula 1) is 100:0.01 to 1 and preferably 100:0.01 to 0.5 in the dental curable composition, allows favorable long-term stability thereof to be expected.

In the case of a combination of the pretreatment method and the integral blend method, the above-described compounding ratio can be calculated from a sum of the compounding ratios in each case. Even in this case, using a filler which has been surface-treated so that a compounding ratio of the (C1) component and the compound of formula 1 (component (C1):compound of formula 1) is 100:0.01 to 1 and preferably 100:0.01 to 0.5 in the dental curable composition, allows favorable long-term stability to be expected.

In a case in which surface treatment is carried out in the integral blend method, it is preferable not to contain an inorganic filler that is not hydrophobized other than the (C1) component. This case may make it difficult to control a degree of surface treatment of the (C1) component. On the other hand, in a case in which a filler that has been hydrophobized such as the (C2) component, is contained, the dental curable composition of the present invention exhibits a desired effect, so that an effect on other inorganic fillers can be practically ignored and the degree can be calculated, assuming that surface treatment of the (C1) component is prioritized. Also, in the present invention, surface treatment with a hydrophobic silane coupling agent not only refers to occurrence of irreversible chemical bonding between the inorganic filler and the silane coupling agent, but also refers to any interaction such as a reversible chemical bond or physical adsorption.

The (C) filler used in the present invention may be coated with polysiloxane and used as polysiloxane-coated filler.

A method for producing the polysiloxane-coated filler will be specifically described. A silane compound represented by formula 2 is mixed into an aqueous dispersion containing a filler that has undergone micropulverization to a desired primary particle size (D50) by pulverization or the like, and the silane compound is hydrolyzed or partially hydrolyzed in the system to form a silanol compound, which is then condensed to form polysiloxane, with which a filler surface is then coated to form a polysiloxane-coated filler. It is to be noted that the layer formed when the filler has been surface-treated with the silane compound represented by formula 2 is a polysiloxane-coated layer, which has a hydrophilic surface, so that in the present invention, the silane compound represented by formula 2 is distinguished from the hydrophobic silane coupling agent.

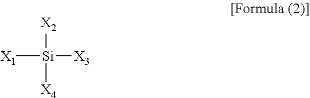

wherein X₁ to X₄ are hydroxyl groups, halogen groups, or alkoxy groups having 8 or less carbon atoms, and X₁ to X₄ may be the same or different.

Specific examples of the silane compound represented by formula 2 include one or more selected from tetramethoxysilane, tetraethoxysilane, tetrapropoxysilane, tetrabutoxysilane, tetrakis(2-ethylhexyloxy)silane, trimethoxychlorosilane, triethoxychlorosilane, triisopropoxychlorosilane, trimethoxyhydroxysilane, diethoxydichlorosilane, tetraphenoxysilane, tetrachlorosilane, or silicon hydroxide (silicon oxide hydrate), and more preferably tetramethoxysilane and/or tetraethoxysilane.

Also, the silane compound is more preferably a low condensate of the silane compound represented by formula 2. For example, it is a low condensate of a silane compound obtained by partially hydrolyzing and condensing tetramethoxysilane and/or tetraethoxysilane. These compounds may be used singly or in combination thereof.

The (C) filler used herein may be surface-treated with an acidic compound. In particular, it is preferable that the (C1) inorganic filler having X-ray radiopacity is surface-treated with an acidic compound. Surface-treating the filler with an acidic compound makes the dental curable composition less stringiness when discharged from a bottle container, resulting in a dental curable composition with a feeling of sharpness upon discharge.

A method for surface-treating the (C) filler with an acidic compound will be exemplified. It is preferable to surface-treat the (C) filler with an acidic compound on the polysiloxane-coated filler. For treatment with the acidic compound, any equipment commonly used in the industry can be used as long as it is a dry fluidized mixer, and examples of such equipment include a Henschel mixer, a super mixer, and a high-speed mixer. The treatment of (C) filler with the acidic compound can be carried out by bringing the filler into contact with a solution containing an acidic compound by impregnation, spraying, or the like. For example, the (C) filler is fluidized in a dry atmosphere, and while it is maintained in the fluidized state, a solution containing an acidic compound is dispersed from above and the mixture is only thoroughly stirred. In this case, a method for dispersing the solution containing the acidic compound is not particularly limited, however, a dropping or spraying method capable of uniformly dispersing the solution is preferable.

After the dispersion step of the solution containing the acidic compound and the (C) filler, it is preferable to remove water and an organic solvent contained therein. A heat treatment method for removing the water and organic solvent is not particularly limited, and can be carried out by a known general method. A piece of equipment used for the heat treatment is preferably a box-type hot air dryer or the like, or a rotary heat treatment apparatus capable of uniform heating or the like. A heat treatment temperature is in the range of room temperature to 200° C. and more preferably in the range of 40 to 150° C. When the temperature is lower than this range, an aqueous medium is not sufficiently removed, and when it is higher than this range, the acidic compound may be decomposed and discolored. Since a heat treatment time depends on the capacity of the dryer, and the like, it is of no problem as long as the aqueous medium can be sufficiently removed. After the heat treatment, the heat-treated product can be easily crushed by applying shear force or impact force, and a crushing method can be performed using the equipment used in the above reaction.

A solvent used for preparing a solution containing an acidic compound used in the reaction can be any solvent as long as the acidic compound is dissolved, and examples thereof include one or more selected from water, ethanol, or acetone. Among these, one or more selected from water or ethanol is particularly preferred. The amount of acidic compound added relative to 100 parts by mass of the (C) filler is preferably in the range of 0.5 to 10 parts by mass.

Examples of the acidic compound used in the treatment with an acidic compound include one or more selected from an inorganic acid such as phosphoric acid or hydrochloric acid, an organic acid such as acetic acid, the (A1) polymerizable monomer having an acidic group, or an acidic polymer. Among these, the acidic polymer can be most preferably used. The acidic polymer is a copolymer or a homopolymer of a polymerizable monomer having, as an acidic group, one or more acidic groups selected from a phosphoric acid residue, a pyrophosphoric acid residue, a thiophosphoric acid residue, a carboxylic acid residue, or a sulfonic acid group, and specific examples of these polymerizable monomers include one or more selected from acrylic acid, methacrylic acid, 2-chloroacrylic acid, 3-chloroacrylic acid, aconitic acid, mesaconic acid, maleic acid, itaconic acid, fumaric acid, glutaconic acid, citraconic acid, 4-(meth)acryloyloxyethoxycarbonylphthalic acid, 4-(meth)acryloyloxyethoxycarbonylphthalic anhydride, 5-(meth)acryloylaminopentylcarboxylic acid, 11-(meth)acryloyloxy-1,1-undecanedicarboxylic acid, 2-(meth)acryloyloxyethyl dihydrogen phosphate, 10-(meth)acryloyloxydecyl dihydrogen phosphate, 20-(meth)acryloyloxyeicosyl dihydrogen phosphate, 1,3-di(meth)acryloyloxypropyl-2-dihydrogen phosphate, 2-(meth)acryloyloxyethyl phenyl phosphate, 2-(meth)acryloyloxyethyl-2′-bromoethyl phosphate, (meth)acryloyloxyethyl phenyl phosphonate, di(2-(meth)acryloyloxyethyl) pyrophosphate, 2-(meth)acryloyloxyethyl dihydrogen dithiophosphate, or 10-(meth)acryloyloxydecyl dihydrogen thiophosphate. Among these, it is preferable to use a homopolymer or a copolymer of an α-β unsaturated carboxylic acid, and more specifically, one or more selected from an acrylic acid polymer, an acrylic acid-maleic acid copolymer, or an acrylic acid-itaconic acid copolymer can be preferably used.

The preferred weight-average molecular weight of the acidic polymer is in the range of 2,000 to 50,000 and preferably in the range of 5,000 to 40,000. When treated with an acidic polymer having a weight-average molecular weight of 2,000 or more, the dental curable composition tends to have less stringiness when discharged from a bottle container and tends to have a favorable feeling of sharpness upon discharge, and when treated with an acidic polymer having a weight-average molecular weight of 50,000 or less, the viscosity of the solution containing an acidic polymer is lower, facilitating surface treatment.

The (C1) inorganic filler having X-ray radiopacity contained in the dental curable composition of the present invention is preferably contained in an amount of 40 to 120 parts by mass relative to 100 parts by mass of the (A) polymerizable monomer contained in the dental curable composition. In the case of the amount compounded of less than 40 parts by mass, sufficient X-ray radiopacity may not be exhibited, and in the case of the amount compounded of more than 120 parts by mass, appropriate flowability may not be exhibited and operability may be deteriorated when the dental curable composition has been put in a bottle container.

[(C2) Hydrophobized Silica Fine Particle Having a Primary Particle Size of Less than 0.1 μm]

The dental curable composition of the present invention contains a (C2) hydrophobized silica fine particle having a primary particle size of less than 0.1 μm (also referred to as “(C2) component” in the present invention). Containing the hydrophobized silica fine particle enables the inorganic filler having X-ray radiopacity to be inhibited from precipitation while maintaining favorable operability of the dental curable composition even when it is filled into a bottle container.

Specific examples of a hydrophobization method for the hydrophobized silica fine particle include a surface treatment method using modified silicone oil such as dimethyl silicone oil, and/or a surface treatment method using a silane coupling agent having one or more functional groups selected from a trimethylsilyl group, a dimethylsilyl group, a methylsilyl group, or an alkylsilyl group that may have a (meth)acryloyl group with an alkyl chain having 3 or more and 18 or less carbon atoms. The silane coupling agent preferably hydrophobizes the silica fine particle via a covalent bond.

Specific examples of the modified silicone oil and silane coupling agent include polydimethylsiloxane, hexamethyldisilazane, dimethylpolysiloxane, methylchlorosilane, alkylalkoxysilanes such as alkyltrialkoxysilane, dialkyldialkoxysilane, octadecylalkoxysilane and octylalkoxysilane, and (meth)acryloylalkylalkoxysilanes such as 3-methacryloylpropyltrimethoxysilane and 8-methacryloyloctyltrimethoxysilane.

In order to further improve rheological properties, the silica fine particle may be hydrophobized by a plurality of methods. For example, the silica fine particle may be treated with a surface treatment agent after having been treated with a silane coupling agent and/or modified silicone oil or may be treated simultaneously.

Examples of the (C2) hydrophobized silica fine particle having a primary particle size of less than 0.1 μm include dry silica, silica aerogel, and wet silica, with dry silica being more preferred.

Among the hydrophobized silica fine particles described above, the dry silica is produced and sold under the trade name Aerosil by, for example, Nippon Aerosil Co., Ltd. Silica fine powders that have undergone hydrophobization surface treatment, such as Aerosil 50, Aerosil 90, Aerosil 130, Aerosil 200, Aerosil 300, Aerosil 380, Aerosil OX50, Aerosil TT600, furthermore, Aerosil R972, Aerosil R974, Aerosil R976, Aerosil R976S, Aerosil R202, Aerosil R812, Aerosil R812S, Aerosil R805, Aerosil R104, Aerosil R106, RY200, RX200, R711, RY200S, RA200H, R8200, and RA200HS, may be used.

The amount compounded of (C2) hydrophobized silica fine particle having a primary particle size of less than 0.1 μm is 3 to 20 parts by mass and preferably 5 to 15 parts by mass, relative to 100 parts by mass of (A) polymerizable monomer. The amount of 3 parts by mass or more relative to 100 parts by mass of (A) polymerizable monomer tends to make the (C) filler contained in the dental curable composition less likely to be precipitated, and the amount of 20 parts by mass or less tends to result in a dental curable composition with favorable operability.

The primary particle size in the present invention refers to an average primary particle size. The primary particle size of the (C) filler can be an average calculated based on a particle distribution measured by, for example, a laser diffraction particle size distribution measuring apparatus. For example, it can be measured by a laser diffraction particle size measuring apparatus (Microtrac MT3300EXII, manufactured by Nikkiso Co., Ltd.). When measured using a laser diffraction particle size measuring apparatus, the particle is measured after having been dispersed by ultrasonic waves or the like so that the filler is uniform. In addition thereto, a primary particle size can be measured by dynamic light scattering particle size measurement or by using an electron microscope photograph in a case where a primary particle is strongly aggregated to form a secondary particle. For a filler having a primary particle size of less than 0.1 μm, it is preferable to calculate the primary particle size from an electron microscope photograph, and for a filler having a primary particle size of 0.1 μm or more, it is preferable to calculate the primary particle size using a laser diffraction particle size distribution measuring apparatus.

The type of (C) filler is not limited as long as it is a known filler, and a filler according to its application can be compounded, and it is preferable to compound a filler such as an inorganic filler, an organic filler, an organic-inorganic composite filler, or an ion-sustained release glass. The dental curable composition of the present invention may use the exemplified fillers singly or in combination of two or more thereof.

### <Other Component>

Also, the dental curable composition of the present invention may contain a component other than the above-described components (A) to (C) as long as effects of the present invention are not impaired. For example, benzophenone-based and benzotriazole-based ultraviolet absorbers, an α-alkylstyrene compound, mercaptan compounds such as n-butyl mercaptan and n-octyl mercaptan, chain transfer agents such as terpenoid-based compounds such as limonene, myrcene, α-terpinene, β-terpinene, γ-terpinene, terpinolene, β-pinene, and α-pinene, metal scavengers such as an aminocarboxylic acid-based chelating agent and a phosphonic acid-based chelating agent, a discoloration inhibitor, a polymerization inhibitor, an antibacterial agent, a color pigment, and other conventionally known additives, can be arbitrarily added, if necessary.

A method for producing the dental curable composition of the present invention is not particularly limited. An example of a general method for producing the dental curable composition includes a method for fabricating a matrix obtained by preliminarily mixing the (A) polymerizable monomer, the (B) polymerization initiator, and the like, excluding the (C) filler by a known method such as a planetary centrifugal mixer, a tumbler mixer, a mix rotor, a dissolver, or a planetary mixer, then kneading the matrix and the (C) filler by a known method such as a planetary centrifugal mixer, a tumbler mixer, a mix rotor, a dissolver, or a planetary mixer, and removing air bubbles under reduced pressure to prepare uniform paste. The matrix used herein refers to a mixed solution fabricated by mixing the components excluding the (C) filler, and specifically refers to a mixed solution fabricated by preliminarily mixing the (A) polymerizable monomer, the (B) polymerization initiator, and the like. In a case in which either one or more of the (A) polymerizable monomer and the (B) polymerization initiator is not uniformly dissolved in the matrix, a matrix free of one or more thereof can also be prepared. As a method for compounding the (A) polymerizable monomer and (B) polymerization initiator, which are not contained in a matrix into the dental curable composition, it is preferable to compound them upon mixing of the matrix and (C) filler. An example of a component undissolvable in the matrix includes a component undissolvable even when mixed for 48 hours under the condition of 100 rpm using a mix rotor. In this case, for a component that can be mixed at high temperatures, it is preferable to mix the component at a temperature of 50° C. On the other hand, for a component that may be deteriorated due to heating, such as the (B) polymerization initiator, it is preferable to mix the component at a temperature of 20 to 30° C. The most preferable production method is a method for preliminarily mixing the (A) polymerizable monomer and (B) polymerization initiator to prepare a uniform matrix, and then mixing the (C) filler to produce a dental curable composition. In the present invention as well, a dental curable composition can be produced without any problems by the above-described production method.

The dental curable composition of the present invention may contain only the (A) polymerizable monomer, the (B) polymerization initiator, and the (C) filler. Furthermore, the dental curable composition may contain only one or more of the above-described components as components other than the (A) to (C).

The dental curable composition of the present invention can be used as a kit with a dental adhesive composition. More specifically, it is a dental adhesive composition kit that can be used by coating an adherend with the first agent, and then coating the coated adherend with the dental curable composition of the present invention, which is the second agent, without irradiation of light.

The first agent included in the dental adhesive composition kit, contains the (A1) polymerizable monomer having an acidic group. As the (A1) polymerizable monomer having an acidic group which can be suitably used, one or more selected from 10-methacryloyloxydecyl dihydrogen phosphate, 6-methacryloxyhexyl phosphonoacetate, 4-methacryloxyethyl trimellitic acid, or 4-methacryloxyethyl trimellitic anhydride, can be suitably used. The preferred amount compounded of the (A1) polymerizable monomer having an acidic group relative to 100 parts by mass of the composition of the first agent is 2 to 30 parts by mass. The (A1) polymerizable monomer having an acidic group contained in the first agent in an amount compounded of 2 parts by mass or more tends to exhibit favorable adhesive strength, and an amount compounded of 30 parts by mass or less tends to indicate favorable stability.

The first agent included in the dental adhesive composition kit may contain an (A2) polymerizable monomer having no acidic group. Examples of the (A2) polymerizable monomer having no acidic group that can be suitably used include one or more selected from 2-hydroxyethyl(meth)acrylate, 3-hydroxypropyl (meth)acrylate, 2,2-bis[4-(3-(meth)acryloyloxy)-2-hydroxypropoxyphenyl]propane, glycerol dimethacrylate, or triethylene glycol dimethacrylate. The amount compounded of the (A2) polymerizable monomer having no acidic group is preferably 0 to 50 parts by mass relative to 100 parts by mass of the composition of the first agent.

The first agent included in the dental adhesive composition kit contains (D) water (also referred to as “(D) component” in the present invention). Specific examples of the (D) water include one or more selected from deionized water or distilled water. Also, the amount compounded of (D) water is preferably 5 to 50 parts by mass relative to 100 parts by mass of the composition of the first agent. The amount compounded of 5 parts by mass or more tends to result in that adhesive strength to dentin was favorable, and the amount compounded of 50 parts by mass or less may result in that the (A) polymerizable monomer contained in the first agent has favorable solubility.

The first agent contained in the dental adhesive composition kit may contain an (E) volatile organic solvent (also referred to as “(E) component” in the present invention). As the (E) volatile organic solvent, an organic solvent that has a boiling point of 150° C. or less under normal pressure and a degree of solubility in water at 25° C. of preferably 5% by mass or more and more preferably 30% by mass or more, and most preferably an infinite percentage by mass (the organic solvent soluble in water at any ratio), is used. Among these, a water-soluble volatile organic solvent having a boiling point of 100° C. or less under normal pressure is preferred, and specific examples thereof include one or more selected from ethanol, methanol, 1-propanol, isopropyl alcohol, acetone, methyl ethyl ketone, 1,2-dimethoxyethane, 1,2-diethoxyethane, or tetrahydrofuran. Among the above-described volatile organic solvents, one or more selected from ethanol, isopropyl alcohol, acetone, or methyl ethyl ketone are more preferred. Also, the amount compounded of the (E) volatile organic solvent is preferably 0 to 50 parts by mass relative to 100 parts by mass of the composition of the first agent.

### <Other Component>

The first agent contained in the dental adhesive composition kit may contain a component other than the above-described (A1) and (D) components as long as the effects of the present invention are not impaired. For example, a diluting agent represented by fumed silica, benzophenone-based and benzotriazole-based ultraviolet absorbers, an α-alkylstyrene compound, mercaptan compounds such as n-butyl mercaptan and n-octyl mercaptan, chain transfer agents such as terpenoid-based compounds such as limonene, myrcene, α-terpinene, α-terpinene, γ-terpinene, terpinolene, β-pinene, and α-pinene, metal scavengers such as an aminocarboxylic acid-based chelating agent and a phosphonic acid-based chelating agent, a discoloration inhibitor, an antibacterial agent, a color pigment, and other conventionally known additives, can be arbitrarily added, if necessary.

### EXAMPLES

The materials and their abbreviations used in Examples and Comparative Examples are shown below.

### [(A) Polymerizable Monomer]

< (A1) Polymerizable Monomer with an Acidic Group>.
MDP: 10-Methacryloyloxydecyl dihydrogen phosphate
MET: 4-Methacryloxyethyl trimellitic acid
META: 4-Methacryloyloxyethoxycarbonyl phthalic anhydride

### < (A2) Polymerizable Monomer Having No Acidic Group>

BisGMA: 2,2-Bis[4-(3-methacryloyloxy-2-hydroxypropoxy)phenyl]propane
UDMA: N,N-(2,2,4-trimethylhexamethylene)bis[2-(aminocarboxy)ethanol]methacrylate
TEGDMA: Triethylene glycol dimethacrylate
GDMA: Glycerol dimethacrylate
HEMA: Hydroxyethyl methacrylate

### <Hydrophobic Silane Coupling Agent>

MPTMS: (3-Methacryloyloxypropyl) trimethoxysilane
MPTES: (3-Methacryloyloxypropyl) triethoxysilane

### [(B) Polymerization Initiator]

CQ: Camphorquinone.
BAPO: Phenylbis(2,4,6-trimethylbenzoyl) phosphine oxide
DMBE: N,N-Dimethylaminobenzoic acid ethyl ester.
DHPPT: N,N-Di(2-hydroxypropyl)-p-toluidine
DEPT: N,N-Di(2-hydroxyethyl)-p-toluidine

### [(C) Filler]

### < (C1) Inorganic Filler Having X-Ray Radiopacity>

(Filler C1: The Surface-Treated Amount with Hydrophobic Silane Coupling Agent: 0 Parts by Mass)

Fluoroaluminoborosilicate glass (primary particle size 3 μm, SiO₂: 22.5 wt %, Al₂O₃: 20.0 wt %, B₂O₃: 12.3 wt %, SrO: 35.7 wt %, Na₂O: 2.5 wt %, and F: 7.0 wt %) (Filler C2: The surface-treated amount with hydrophobic silane coupling agent: 0 parts by mass)

Fluoroaluminoborosilicate Glass (primary particle size 1 μm, SiO₂: 22.5 wt %, Al₂O₃: 20.0 wt %, B₂O₃: 12.3 wt&, SrO: 35.7 wt %, Na₂O: 2.5 wt %, and F: 7.0 wt %)

(Filler C3: The Surface-Treated Amount with Hydrophobic Silane Coupling Agent: 0 Parts by Mass)

To 100 g of the filler C2 was added 4.5 g of a low condensate of a silane compound “MKC Silicate MS56S” (SiO₂ content 56.0 wt %, degree of polymerization 2 to 100, manufactured by Mitsubishi Chemical Corporation), the mixture was stirred and mixed for approximately 90 minutes. After having mixed for a prescribed time, the resulting treated slurry was aged at 50° C. for 40 hours in a hot air dryer, then raised to a temperature of 150° C. and held for 6 hours, and then cooled to obtain a heat-treated product. The resulting heat-treated product was placed in a Henschel mixer and crushed at 1,800 rpm for 5 minutes to obtain a polysiloxane-coated filler. 16.0 grams of an aqueous solution containing polyacrylic acid, which was an acidic compound (polymer concentration 13% by mass and weight-average molecular weight 10,000, manufactured by NAKARAI TESQUE, INC.) was sprayed onto 100 g of the polysiloxane-coated filler. After spraying, the powder removed from the mixer was heated at 100° C. for 3 hours in a hot air dryer. After the temperature returned to room temperature, the powder was sieved to obtain a filler C3.

(Filler C4: The Surface-Treated Amount with Hydrophobic Silane Coupling Agent: 0.2 Parts by Mass)

To 100 g of the filler C1 was added silane coupling treatment liquid containing 5 g of water, 10 g of ethanol, and 0.2 g of 3-methacryloyloxypropyltrimethoxysilane as a silane coupling agent, and the mixture was stirred and mixed for 2 hours. Thereafter, the mixture was heat-treated at 90° C. for 15 hours, and then underwent a sieving step to obtain a filler C4.

(Filler C5: The Surface-Treated Amount with Hydrophobic Silane Coupling Agent: 0.05 Parts by Mass)

To 100 g of the filler C2 was added silane coupling treatment liquid containing 5 g of water, 10 g of ethanol, and 0.05 g of 3-methacryloyloxypropyltrimethoxysilane as a silane coupling agent, and the mixture was stirred and mixed for 2 hours. Thereafter, the mixture was heat-treated at 90° C. for 15 hours, and then underwent a sieving step to obtain a filler C5.

(Filler C6: The Surface-Treated Amount with Hydrophobic Silane Coupling Agent: 0.1 Parts by Mass)

To 100 g of the filler C3 was added silane coupling treatment liquid containing 5 g of water, 10 g of ethanol, and 0.1 g of 3-methacryloyloxypropyltrimethoxysilane as a silane coupling agent, and the mixture was stirred and mixed for 2 hours. Thereafter, the mixture was heat-treated at 90° C. for 15 hours, and then underwent a sieving step to obtain a filler C6.

(Filler C7: The Surface-Treated Amount with Hydrophobic Silane Coupling Agent: 0.6 Parts by Mass)

To 100 g of the filler C3 was added a silane coupling treatment liquid containing 5 g of water, 10 g of ethanol, and 0.6 g of 3-methacryloyloxypropyltrimethoxysilane as a silane coupling agent, and the mixture was stirred and mixed for 2 hours. Thereafter, the mixture was heat-treated at 90° C. for 15 hours, and then underwent a sieving step to obtain a filler C7.

(Filler C8: The Surface-Treated Amount with Hydrophobic Silane Coupling Agent: 1.5 Parts by Mass)

To 100 g of the filler C1 was added silane coupling treatment liquid containing 5 g of water, 10 g of ethanol, and 1.5 g of 3-methacryloyloxypropyltrimethoxysilane as a silane coupling agent, and the mixture was stirred and mixed for 2 hours. Thereafter, the mixture was heat-treated at 90° C. for 15 hours, and then underwent a sieving step to obtain a filler C8.

(Filler C9: The Surface-Treated Amount with Hydrophobic Silane Coupling Agent: 1.5 Parts by Mass)

To 100 g of the filler C3 was added a silane coupling treatment liquid containing 5 g of water, 10 g of ethanol, and 1.5 g of 3-methacryloyloxypropyltrimethoxysilane as a silane coupling agent, and the mixture was stirred and mixed for 2 hours. Thereafter, the mixture was heat-treated at 90° C. for 15 hours, and then underwent a sieving step to obtain a filler C9.

(C2) a Hydrophobized Silica Fine Particle with a Primary Particle Size of Less than 0.1 μm

### (Filler D21)

Aerosil R-972 (hydrophobized silica surface-treated with dimethyldichlorosilane, primary particle size 16 nm, manufactured by Evonik Industries AG)

### (Filler D22)

Aerosil R-974 (hydrophobized silica surface-treated with dimethyldichlorosilane, primary particle size 12 nm, manufactured by Evonik Industries AG)

### (Filler D23)

Aerosil R-711 (hydrophobized silica surface-treated with a methacrylic-silyl compound, primary particle size 12 nm, manufactured by Evonik Industries AG)

### [(D) Water]

D. W.: Distilled water

### [(E) Volatile Organic Solvent]

EtOH: Ethanol
Acetone: Acetone

### [Other Component]

### [Polymerization Inhibitor]

MeHQ: p-Methoxyphenol
BHT: Dibutylhydroxytoluene

### [Ultraviolet Absorber]

OB: 2-Hydroxy-4-(octyloxy)benzophenone

### [Fluorescent Agent]

FA: Diethyl 2.5-dihydroxyterephthalate

### Production Example 2-1: Production Method of a Dental Curable Composition (Second Agent) (2-1)

A wide-mouthed plastic container was charged with all components except for the (C) filler shown in Table 1, and the mixture was mixed for 48 hours under the condition of 100 rpm using a mix rotor VMRC-5 to obtain a matrix. Thereafter, the matrix and the (C) filler were then fed in a kneading machine, uniformly stirred, and then degassed under vacuum to obtain a dental curable composition (2-1). In the case of use of the dental curable composition as a dental adhesive composition, combined for use with the first agent, it is used as the second agent. It is to be noted that in Tables 1 to 3, the abbreviation for each component are followed by parts by mass of each component in each parenthesis.

### Production Examples 2-2 to 2-18 and Comparative Production Examples 2-1 to 2-4: Production Methods for Dental Curable Compositions (Second Agents) (2-2) to (2-18) and (C2-1) to (C2-4)

Dental curable compositions (2-2) to (2-18) and (C2-1) to (C2-4) were obtained in the similar manner as in Production Example 2-1, except that the compositions in Production Examples 2-2 to 2-18 and Comparative Production Examples 2-1 to 2-4 were changed to those shown in Tables 1 to 3. It is to be noted that the numbers of the production examples correspond to the numbers of the dental curable compositions.

TABLE 1

| Dental | (A) component | | Hydrophobic | | (C) component | | | |
|---|---|---|---|---|---|---|---|---|
| curable | (A1) | (A2) | silane | (B) | (C1) | (C2) | Other | (C1) compo- |
| composition | component | component | coupling agent | component | component | component | component | nent:Formula 1 |
| Production | — | UDMA(50) | — | CQ(1) | C6 (130) | C21 (13) | MeHQ(0.01) | 100:0.1 |
| Example 2-1 | | HEMA(35) | | DMBE(1) | | | | |
| | | TEGDMA(15) | | | | | | |
| Production | — | UDMA(50) | — | CQ(1) | C6 (65) | C21 (13) | OB(1) | 100:0.1 |
| Example 2-2 | | HEMA(35) | | DMBE(1) | | | MeHQ(0.01) | |
| | | TEGDMA(15) | | | | | | |
| Production | — | UDMA(50) | — | CQ(1) | C6 (40) | C22 (10) | OB(1) | 100:0.1 |
| Example 2-3 | | HEMA(35) | | DMBE(1) | | | MeHQ(0.01) | |
| | | TEGDMA(15) | | | | | FA(0.005) | |
| Production | — | UDMA(35) | — | CQ(1) | C6 (40) | C22 (21) | OB(1) | 100:0.1 |
| Example 2-4 | | HEMA(35) | | DMBE(1) | | | MeHQ(0.01) | |
| | | TEGDMA(30) | | | | | FA(0.005) | |
| Production | — | BisGMA(25) | — | CQ(1) | C6 (39) | C22 (3) | OB(1) | 100:0.1 |
| Example 2-5 | | UDMA(15) | | DMBE(1) | | | MeHQ(0.01) | |
| | | HEMA(30) | | DEPT(1) | | | FA(0.005) | |
| | | TEGDMA(30) | | | | | | |
| Production | — | UDMA(50) | — | BAPO | C6 (80) | C21 (12) | MeHQ(0.01) | 100:0.1 |
| Example 2-6 | | HEMA(35) | | (0.45) | | | FA(0.005) | |
| | | TEGDMA(15) | | | | | | |
| Production | — | UDMA(50) | MPTMS | CQ(1.5) | C3 (80) | C21 (10) | MeHQ(0.01) | 100:0.13 |
| Example 2-7 | | HEMA(35) | (0.1) | DMBE(3) | | | FA(0.005) | |
| | | TEGDMA(15) | | DHPPT(1) | | | | |
| Production | — | UDMA(50) | — | CQ(1) | C6 (80) | C21 (13) | OB(1) | 100:0.1 |
| Example 2-8 | | HEMA(35) | | DMBE(1) | | | MeHQ(0.01) | |
| | | TEGDMA(15) | | | | | | |
| Production | — | UDMA(50) | MPTMS | CQ(1) | C1 (80) | C21 (10) | OB(1) | 100:0.13 |
| Example 2-9 | | HEMA(35) | (0.1) | DMBE(1) | | | MeHQ(0.01) | |
| | | TEGDMA(15) | | | | | | |

TABLE 2

| Dental | (A) component | | Hydrophobic | | (C) component | | | |
|---|---|---|---|---|---|---|---|---|
| curable | (A1) | (A2) | silane | (B) | (C1) | (C2) | Other | (C1) compo- |
| composition | component | component | coupling agent | component | component | component | component | nent:Formula 1 |
| Production | — | UDMA(40) | — | CQ(1) | C6 (80) | C21 (12) | MeHQ(0.01) | 100:0.2 |
| Example 2-10 | | GDMA(25) | | DMBE(1) | | | | |
| | | TEGDMA(35) | | DHPPT(1) | | | | |
| Production | MDP | UDMA(47.5) | — | CQ(1) | C3 (80) | C21 (10) | OB(1) | 100:0.05 |
| Example 2-11 | (2.5) | HEMA(35) | | DMBE(1) | | | BHT(0.1) | |
| | | TEGDMA(10) | MPTMS | | | | | |
| Production | — | UDMA(50) | (0.05) | CQ(1) | C6 (80) | C21 (13) | FA(0.005) | 100:0.06 |
| Example 2-12 | | HEMA(35) | | DMBE(1) | | | | |
| | | TEGDMA(15) | | DEPT(1) | | | | |
| Production | — | UDMA(50) | MPTES | CQ(1) | C1 (80) | C21 (10) | — | 100:0.06 |
| Example 2-13 | | HEMA(35) | (0.05) | DMBE(1) | | | | |
| | | TEGDMA(15) | | | | | | |
| Production | — | UDMA(50) | — | CQ(1) | | | OB(0.5) | 100:0.6 |
| Example 2-14 | | HEMA(35) | | DMBE(1) | C4 (80) | C21 (10) | MeHQ(0.01) | |
| | | TEGDMA(15) | | DHPPT(1) | | | | |
| Production | — | UDMA(50) | MPTES | CQ(1) | | | OB(0.5) | 100:0.8 |
| Example 2-15 | | HEMA(35) | (0.8) | DMBE(1) | C5 (80) | C23 (10) | MeHQ(0.01) | |
| | | TEGDMA(15) | | DHPPT(1) | | | | |
| Production | — | UDMA(50) | MPTMS | CQ(1) | | | OB(0.5) | 100:0.03 |
| Example 2-16 | | HEMA(35) | (0.03) | DMBE(1) | C2 (80) | C21 (10) | MeHQ(0.01) | |
| | | TEGDMA(15) | | DHPPT(1) | | | | |
| Production | — | UDMA(50) | MPTMS | CQ(1) | | | OB(1) | 100:0.01 |
| Example 2-17 | | HEMA(35) | (0.01) | DMBE(1) | C3 (80) | C21 (13) | MeHQ(0.01) | |
| | | TEGDMA(15) | | | | | | |
| Production | — | UDMA(50) | | CQ(1) | | | OB(0.5) | 100:0.01 |
| Example 2-18 | | HEMA(35) | | DMBE(1) | C7 (80) | C21 (13) | MeHQ(0.01) | |
| | | TEGDMA(15) | | DEPT(0.5) | | | FA(0.005) | |

TABLE 3

| Dental | (A) component | | Hydrophobic | | (C) component | | | |
|---|---|---|---|---|---|---|---|---|
| curable | (A1) | (A2) | silane | (B) | (C1) | (C2) | Other | (C1) compo- |
| composition | component | component | coupling agent | component | component | component | component | nent:Formula 1 |
| Comparative | — | UDMA(50) | MPTMS | CQ(1) | C6 (80) | C21 (13) | OB(0.5) | 100:2.5 |
| Production | | HEMA(35) | (2) | DMBE(1) | | | MeHQ(0.01) | |
| Example 2-1 | | TEGDMA(15) | | | | | FA(0.005) | |
| Comparative | — | UDMA(50) | — | CQ(1) | C1 (80) | C21 (13) | OB(0.5) | 100:0.00 |
| Production | | HEMA(35) | | DMBE(1) | | | MeHQ(0.01) | |
| Example 2-2 | | TEGDMA(15) | | | | | FA(0.005) | |
| Comparative | — | UDMA(50) | — | CQ(1) | C9 (80) | C21 (13) | OB(0.5) | 100:1.5 |
| Production | | HEMA(35) | | DMBE(1) | | | MeHQ(0.01) | |
| Exemple 2-3 | | TEGDMA(15) | | | | | FA(0.005) | |
| Comparative | — | UDMA(50) | — | CQ(1) | — | C21 (13) | OB(0.5) | — |
| Production | | HEMA(35) | | DMBE(1) | | | MeHQ(0.01) | |
| Example 2-4 | | TEGDMA(15) | | | | | FA(0.005) | |

### Production Example 1-1: Production Method of a First Agent (1-1)

A wide-mouthed plastic container was charged with all components shown in Table 4, and the mixture was mixed for 48 hours under the condition of 100 rpm using a mix rotor VMRC-5 to obtain the first agent (1-1). It is to be noted that in Tables 4, the abbreviation for each component is followed by parts by mass of each component in each parenthesis.

### Production Examples 1-2 to 1-9: Production Methods of First Agents (1-2) to (1-9)

The first agents (1-2) to (1-9) were obtained in the similar manner as in Production Example 1-1, except that the compositions in Production Examples 1-2 to 1-9 were changed to those shown in Table 4. It is to be noted that the numbers of the production examples correspond to the numbers of the first agents.

TABLE 4

| | | | | (C) com- | | | |
|---|---|---|---|---|---|---|---|
| Dental | (A) component | | | ponent | (D) | (E) | Other |
| adhesive | (A1) | (A2) | (B) | (C2) com- | compo- | compo- | compo- |
| composition | component | component | component | ponent | nent | nent | nent |
| Production | MDP(15) | HEMA(35) | CQ(0.5) | — | D.W. | — | BHT |
| Example | | | DMBE(0.4) | | (50) | | (0.1) |
| 1-1 | | | | | | | |
| Production | MDP(10) | — | CQ(0.1) | — | D.W. | EtOH | BHT |
| Example | META(20) | | DMBE(0.3) | | (30) | (40) | (0.1) |
| 1-2 | | | | | | | |
| Production | MDP(10) | — | CQ(0.1) | — | D.W. | Acetone | BHT |
| Example | META(20) | | DMBE(0.3) | | (5) | (50) | (0.1) |
| 1-3 | | | | | | | |
| Production | MDP(15) | HEMA(20) | CQ(0.5) | — | D.W. | Acetone | BHT |
| Example | | BisGMA(15) | DMBE(0.4) | | (15) | (20) | (0.2) |
| 1-4 | | GDMA(15) | | | | | |
| Production | MDP(15) | HEMA(15) | CQ(0.5) | C23 | D.W. | EtOH | BHT |
| Example | | BisGMA(15) | DMBE(0.4) | (10) | (15) | (40) | (0.2) |
| 1-5 | | | | | | | |
| Production | MDP(15) | HEMA(10) | CQ(0.5) | — | D.W. | Acetone | BHT |
| Example | | BisGMA(15) | DMBE(0.4) | | (20) | (30) | (0.2) |
| 1-6 | | TEGDMA(10) | | | | | |
| Production | MDP(10) | — | DMBE(0.4) | — | D.W. | EtOH | BHT |
| Example | META(20) | | | | (30) | (40) | (0.2) |
| 1-7 | | | | | | | |
| Production | MDP(10) | — | — | — | D.W. | EtOH | BHT |
| Example | MET(15) | | | | (35) | (40) | (0.2) |
| 1-8 | | | | | | | |
| Production | MDP(2) | HEMA (20) | CQ(0.5) | — | D.W. | EtOH | BHT |
| Example | | | DMBE(0.5) | | (50) | (28) | (0.2) |
| 1-9 | | | | | | | |

### <Evaluation 1: X-Ray Radiopacity>

The prepared dental curable composition was filled into a stainless steel mold (15 φ×1 mm: disk-shaped), a cover glass was then placed from above, and the dental curable composition was pressed with the glass plate. The dental curable composition was irradiated with light from above the cover glass for 1 minute using a photopolymerization irradiator (Grip Light II, manufactured by Matsukaze Co., Ltd.) to cure the dental curable composition, and the cured product was removed from the mold and the cover glass was then removed. An X-ray transmission image was acquired using an aluminum step wedge as well as an X-ray transmission inspection apparatus (3MX-31M, manufactured by Shimadzu Corporation).

The obtained X-ray transmission image was analyzed using image analysis software ImageJ. A calibration curve of a gray value for each thickness of the aluminum step wedge was created, and a ratio of a gray value of the dental curable composition to that of aluminum having a thickness of 1 mm was taken as X-ray radiopacity of the dental curable composition. When the degree of the X-ray radiopacity relative to that of 1 mm of aluminum was 1.0 times or more, it was determined to indicate sufficient X-ray radiopacity and rated A, when it was 0.7 times or more but less than 1.0 times, it was determined to indicate an ordinary level of X-ray radiopacity and rated B, when it was 0.5 times or more but less than 0.7 times, it was determined to indicate low X-ray radiopacity and rated C, and when it was less than 0.5 times, it was determined to indicate extremely low X-ray radiopacity and rated D. Since the dental curable composition having X-ray radiopacity assists follow-up observation of repair site and diagnosis of secondary caries, it preferably has higher X-ray radiopacity.

### <Evaluation 2: Flowability>

0.05 grams of an initially prepared product of the dental curable composition or an accelerated test sample fabricated by having left it stand in a thermostat set at 50° C. for 2 weeks were placed in a lump on a horizontally placed slide glass (soda glass, 70 mm×70 mm) and within 5 seconds, the slide glass was fixed vertically at 90° to the horizontal plane, and after 1 minute, a distance moved by the dental curable composition is measured. This movement distance (unit: millimeters) is taken as flowability. Flowability with a movement distance of 15 mm or more was determined to be particularly favorable high-flowability. Flowability with a movement distance of 10 mm or more and less than 15 mm was determined to be favorable high-flowability. Flowability with a movement distance of less than 10 mm was determined not to be high-flowability. The initially prepared product and the accelerated test sample having a change in distance of less than 5 mm are determined to have particularly excellent storage stability, and rated A, those having a change of 5 mm or more but less than 10 mm were determined to have average storage stability and rated B, those having a change of 10 mm or more were determined to have low storage stability, and rated D. Since the dental curable composition of the present invention is put in a bottle container and used, it is preferably a highly flowable composition. Also, no change in flowability after long-term storage can expect operability that has been always pursued, which is therefore preferred.

### <Evaluation 3: Favorableness of Dischargeability>

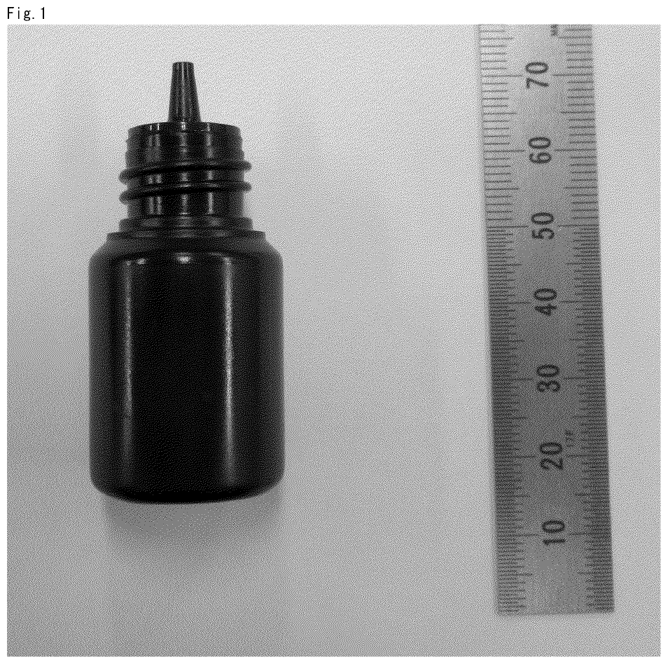

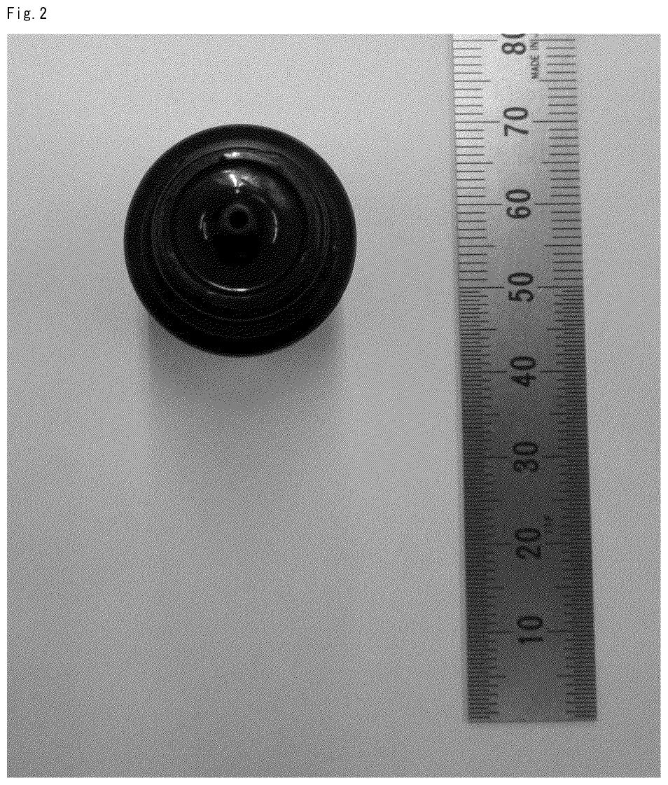

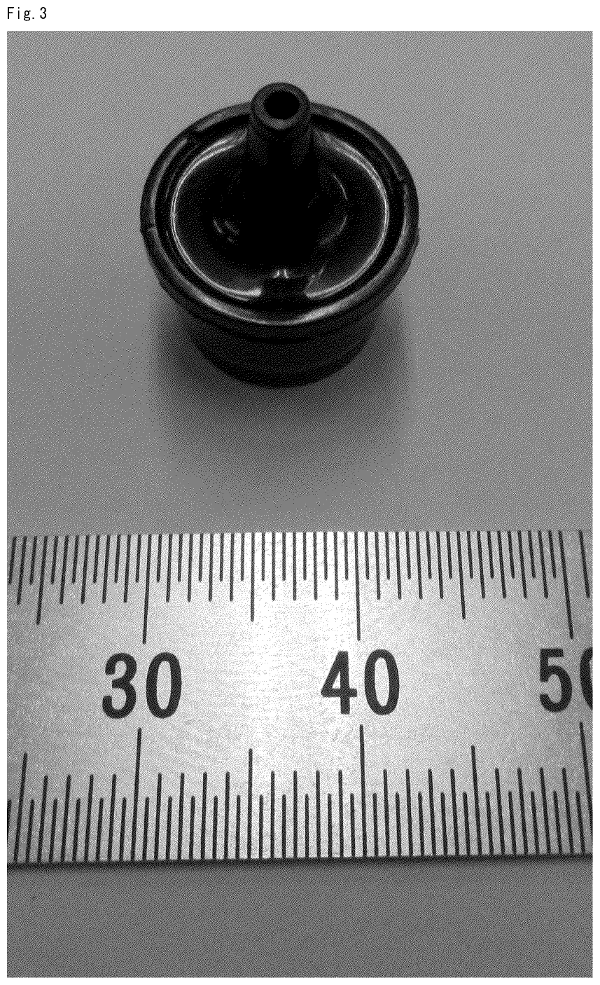

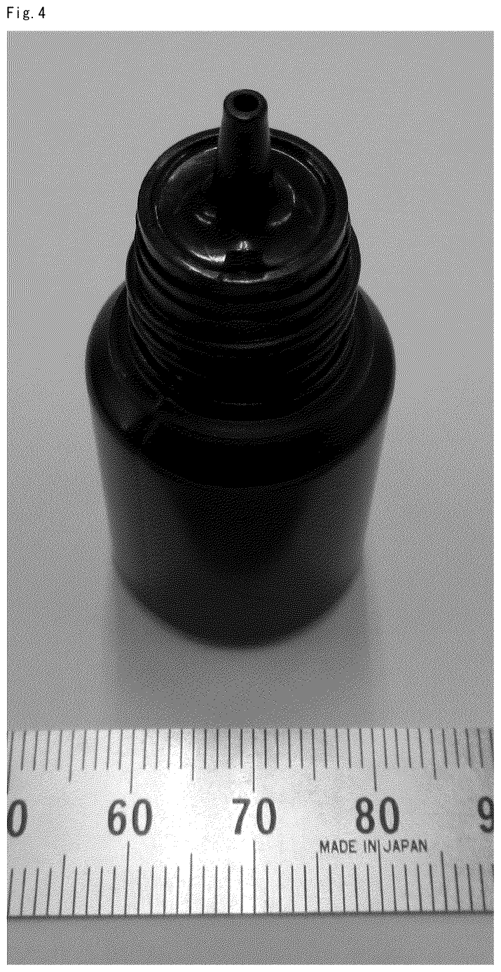

5 grams of the dental curable composition of Example or Comparative Example were filled into a container for glue (FIGS. 1 to 4, nozzle diameter 3 mm, height 51 mm, width 22 mm). After having been filled, the container for glue was left to stand for one week, and a feeling upon discharge was evaluated based on the time it took for the dental curable composition to be discharged from the nozzle when the container for glue was made upside down. The time it took to be discharged being within 30 seconds, was rated A, which was regarded as a favorable feeling upon discharge; the time of 30 to 60 seconds was rated B, which was a normal feeling upon discharge; and the time exceeding 60 seconds, was rated C, which was a poor feeling upon discharge. In a case in which the dental curable composition had too strong a thixotropy or was highly viscous, a feeling upon discharge after having left it stand for one week tends to become poor. A favorable feeling upon discharge means that the dental curable composition can be quickly discharged from a bottle and used when the user wants to use it, which is preferred.

### <Evaluation 4: Appearance Confirmation (Confirmation of Exudate)>

A 50 mL light-shielding container was fed with 30 g of the dental curable composition of Example or Comparative Example and stored in a thermostatic chamber at 50° C. After storage in the thermostatic chamber at 50° C. for one or two weeks, an appearance of the light-shielding container was confirmed. When the dental curable composition caused no exudate, and was not precipitated even after two weeks of storage at 50° C. and maintained a uniform state, it was determined to have favorable stability, which was rated A. When the dental curable composition caused no exudate and was not precipitated after one week of storage at 50° C., and maintained a uniform state, however, it caused exudate, or was precipitated after two weeks of storage at 50° C., and did not maintain a uniform state, the dental curable composition was determined to have an ordinary level of stability, which was rated B. The dental curable composition which was separated and/or precipitated after one week of storage at 50° C., was determined to have poor storage stability, which was rated D. When the dental curable composition is not sufficiently homogeneous, an operation for rendering it homogeneous is required each time the composition is used, resulting in a burden on the user, which is not preferable.

### <Evaluation 5: Adhesive Strength>

A test piece of a bovine central incisor embedded in an epoxy resin was polished with waterproof abrasive paper #500 to shave a dentin surface. Thereafter, an adherent surface was coated with the first agent described in Example and air-dried. Next, a surface of the first agent was coated with the second agent, and was irradiated with light for 10 seconds using an LED light irradiator for dental polymerization (PEN Bright, manufactured by Matsukaze Co., Ltd.). Thereafter, a mold of 2.38 mm @ with a hole (manufactured by Ultradent Products, Inc.) was placed on top, and a flowable composite resin for dental filling (Beautyfil Flow Plus X, manufactured by Matsukaze Co., Ltd.) was filled into the mold, and was irradiated with light for 10 seconds using an LED light irradiator for dental polymerization (PEN Bright, manufactured by Matsukaze Co., Ltd.). The mold was removed, and a procedure of immersing the fabricated test specimen in 37° C. water for 24 hours, and then immersing in a 4° C. cold water phase and a 60° C. hot water phase for 30 seconds each, using a thermal shock tester (manufactured by Thomas Kagaku Co., Ltd.), was repeated 5,000 times. Thereafter, using a universal testing machine (manufactured by Instron Corporation), the test specimen fabricated was then measured for shear adhesive strength at a crosshead speed of 1 mm/min. Adhesive strength of 20 MPa or more was determined to be extremely favorable. Adhesive strength of 10 MPa or more but less than 20 MPa was determined to be of an ordinary level of adhesive strength, and adhesive strength of less than 10 MPa was determined to be of insufficient adhesive strength. Since the adhesive strength is related to long-term stability of a repair site, the higher the strength is preferred.

The results of each test shown in Tables 5 to 7 will be described below.

TABLE 5

| | | | Flowability | | Favorable- | | Adhesive strength of | |
|---|---|---|---|---|---|---|---|---|
| | | X-ray | Measure- | Long- | ness of | Presence or | initially prepared | |
| | Second | radio- | ment | term | discharge | absence of | product | |
| | agent | pacity | value | stability | ability | exudate | First agent | Dentin |
| Example 1 | Production | A | 11 | A | B | A | Production | 17 |
| | Example | | | | | | Example | |
| | 2-1 | | | | | | 1-1 | |
| Example 2 | Production | B | 25 | A | A | A | Production | 24 |
| | Example | | | | | | Example | |
| | 2-2 | | | | | | 1-2 | |
| Example 3 | Production | C | 21 | A | A | A | Production | 25 |
| | Example | | | | | | Example | |
| | 2-3 | | | | | | 1-3 | |
| Example 4 | Production | C | 12 | A | B | A | Production | 15 |
| | Example | | | | | | Example | |
| | 2-4 | | | | | | 1-4 | |
| Example 5 | Production | C | 22 | A | A | B | Production | 25 |
| | Example | | | | | | Example | |
| | 2-5 | | | | | | 1-5 | |
| Example 6 | Production | A | 20 | A | A | A | Production | 12 |
| | Example | | | | | | Example | |
| | 2-6 | | | | | | 1-6 | |
| Example 7 | Production | A | 22 | A | A | A | Production | 24 |
| | Example | | | | | | Example | |
| | 2-7 | | | | | | 1-6 | |
| Example 8 | Production | A | 22 | A | A | A | Production | 24 |
| | Example | | | | | | Example | |
| | 2-8 | | | | | | 1-6 | |
| Example 9 | Production | A | 21 | A | B | A | Production | 25 |
| | Example | | | | | | Example | |
| | 2-9 | | | | | | 1-7 | |

TABLE 6

| | | | Flowability | | Favorable- | | Adhesive strength of | |
|---|---|---|---|---|---|---|---|---|
| | | X-ray | Measure- | Long- | ness of | Presence or | initially prepared | |
| | Second | radio- | ment | term | discharge- | absence of | product | |
| | agent | pacity | value | stability | ability | exudate | First agent | Dentin |
| Example | Production | A | 20 | A | B | A | Production | 24 |
| 10 | Example | | | | | | Example | |
| | 2-10 | | | | | | 1-8 | |
| Example | Production | A | 11 | A | B | A | Production | 24 |
| 11 | Example | | | | | | Example | |
| | 2-11 | | | | | | 1-8 | |
| Example | Production | A | 21 | A | B | A | Production | 25 |
| 12 | Example | | | | | | Example | |
| | 2-12 | | | | | | 1-6 | |
| Example | Production | A | 22 | A | A | A | Production | 25 |
| 13 | Example | | | | | | Example | |
| | 2-13 | | | | | | 1-6 | |
| Example | Production | A | 20 | A | A | B | Production | 25 |
| 14 | Example | | | | | | Example | |
| | 2-14 | | | | | | 1-6 | |
| Example | Production | A | 20 | A | A | B | Production | 25 |
| 15 | Example | | | | | | Example | |
| | 2-15 | | | | | | 1-6 | |
| Example | Production | A | 19 | B | A | B | Production | 24 |
| 16 | Example | | | | | | Example | |
| | 2-16 | | | | | | 1-6 | |
| Example | Production | A | 17 | B | A | A | Production | 12 |
| 17 | Example | | | | | | Example | |
| | 2-17 | | | | | | 1-9 | |
| Example | Production | A | 21 | A | A | A | Production | 25 |
| 18 | Example | | | | | | Example | |
| | 2-18 | | | | | | 1-6 | |

TABLE 7

| | | | Flowability | | Favorable- | | Adhesive strength of | |
|---|---|---|---|---|---|---|---|---|
| | | X-ray | Measure- | Long- | ness of | Presence or | initially prepared | |
| | Second | radio- | ment | term | discharge- | absence of | product | |
| | agent | pacity | value | stability | ability | exudate | First agent | Dentin |
| Comparative | Comparative | A | 21 | A | A | D | Production | 24 |
| Example 1 | Production | | | | | | Example | |
| | Example 2-1 | | | | | | 1-2 | |
| Comparative | Comparative | A | 20 | D | A | A | Production | 26 |
| Example 2 | Production | | | | | | Example | |
| | Example 2-2 | | | | | | 1-2 | |
| Comparative | Comparative | A | 21 | A | A | D | Production | 26 |
| Example 3 | Production | | | | | | Example | |
| | Example 2-3 | | | | | | 1-2 | |
| Comparative | Comparative | D | 22 | A | A | A | Production | 24 |
| Example 4 | Production | | | | | | Example | |
| | Example 2-4 | | | | | | 1-2 | |

The compositions described in Examples were confirmed to have X-ray radiopacity and high flowability, as well as favorable operability and excellent stability even when put in a bottle container.

Example 1 with a larger amount compounded of (C1) component, exhibited low flowability and had a likelihood of slightly low adhesiveness to dentin. Also, Examples 4 and 5 with a small amount compounded of inorganic filler containing the (C1) component, tended to have low X-ray radiopacity. Furthermore, Example 4 with a large amount compounded of (C2) component, tended to have low flowability. Examples 14 and 15 with a large amount of (C1) component surface treated with the silane coupling agent of formula 1, tended to facilitate separation. Also Examples 9 to 12, in which the (C1) component not surface-treated with an acidic compound, was used, tended to have poorer dischargeability than the examples (for example, Example 8 and the like) that used the (C1) component surface-treated with an acidic compound.

Example 6 with a small amount compounded of (B) component, tended to have slightly low adhesive strength to dentin. Also, Example 17 in which Production Examples 1 to 9 with a small amount of (A1) component contained in the dental adhesive composition and combined for use upon the dentin adhesion test, were adopted, tended to have low adhesiveness to dentin.

Example 11 in which the dental curable composition contained the (A1) component, tended to have low flowability.

Comparative Examples 1 and 3 with an excessive amount of (C1) component surface treated with the silane coupling agent of formula 1, caused a significant amount of exudate, resulting in the dental curable composition having poor storage stability. Comparative Example 2 without the (C1) component surface treated with the silane coupling agent of formula 1 resulted in a dental curable composition having poor stability in flowability. Comparative Example 4 without the (C1) component, did not exhibit X-ray radiopacity.

Using a composition in which the polymerization initiator was removed from the composition of Example 18, a dental adhesive composition in which the (B) polymerization initiator was not contained in the second agent, was fabricated (not shown in the table), and an attempt to cure it in the same manner as in other Examples and Comparative Examples, was made, however, it was not cured.

The dental curable compositions evaluated in Examples can be used for dental adhesives, dental composite resins, dental core construction materials, dental resin cement, dental coating materials, dental pit and fissure sealants, dental manicure materials, materials for dental 3D printers, materials for orthodontics, and the like. More preferably, the dental curable composition can be used as a dental adhesive composition kit of two-step type containing the first agent and the second agent.

### INDUSTRIAL APPLICABILITY

According to the present invention, the dental curable composition has high flowability while having X-ray radiopacity and achieve both favorable operability and stability even when placed in a bottle container.

1. A dental curable composition, comprising an (A) polymerizable monomer, a (B) polymerization initiator, and a (C) filler,
wherein the (C) component comprises
a (C1) inorganic filler having X-ray radiopacity and
a (C2) hydrophobized silica fine particle having a primary particle size of less than 0.1 μm,
wherein the (C1) component is surface-treated with a hydrophobic silane coupling agent of formula 1, and
a mass ratio of the (C1) component and formula 1 ((C1) component:compound of formula 1) is 100:0.01 to 1,
wherein flowability, which is a distance moved by the dental curable composition with an elapsed time of one minute after 0.05 g of the dental curable composition is placed in a lump on a horizontally placed slide glass plane, and the slide glass is fixed vertically to the horizontal plane, is 10 mm or more. 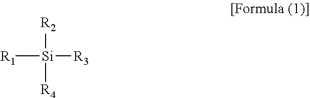
wherein R₁ is an alkyl group having 1 to 11 carbon atoms, which may have one or more selected from a halogen, an aromatic ring, an aliphatic ring, a polymerizable group, —O—, —S—, —NH—, —C(O)—O—, —O—C(O)—, —C(O)—NH—, —O—C(O)—NH—, or —NH—C(O)—O—, and R₂, R₃, and R₄ represent an alkoxy group or an alkyl group having 1 to 4 carbon atoms, or a halogen, and may be the same or different.

2. The dental curable composition according to claim 1, wherein the (C1) inorganic filler having X-ray radiopacity is aluminosilicate glass comprising one or more elements selected from strontium, barium, lanthanum, zirconium, ytterbium, or gadolinium.

3. The dental curable composition according to claim 1, wherein the (C1) inorganic filler having X-ray radiopacity is surface-treated with an acidic compound and a silane coupling agent.

4. The dental curable composition according to claim 2, wherein the (C1) inorganic filler having X-ray radiopacity is surface-treated with an acidic compound and a silane coupling agent.

5. The dental curable composition according to claim 1, wherein the amount of the (C1) inorganic filler having X-ray radiopacity surface-treated with a silane coupling agent is 0.01 to 0.50 parts by mass relative to 100 parts by mass of the (C1) inorganic filler having X-ray radiopacity.

6. The dental curable composition according to claim 2, wherein the amount of the (C1) inorganic filler having X-ray radiopacity surface-treated with a silane coupling agent is 0.01 to 0.50 parts by mass relative to 100 parts by mass of the (C1) inorganic filler having X-ray radiopacity.

7. A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of an (A1) polymerizable monomer having an acidic group,
0 to 50 parts by mass of an (A2) polymerizable monomer having no acidic group,
5 to 50 parts by mass of (D) water, and
0 to 50 parts by of an (E) volatile organic solvent, and
wherein the dental adhesive composition consists of the second agent and the first agent,
wherein the second agent is the second agent according to claim 1, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity, and
3 to 20 parts by mass of the (C2) hydrophobized silica fine particle having a primary particle size of less than 0.1 μm.

8. A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to claim 2, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity, and
3 to 20 parts by mass of the (C2) hydrophobized silica fine particle having a primary particle size of less than 0.1 μm.

9. A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to claim 3, comprising, relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity, and
3 to 20 parts by mass of the (C2) hydrophobized silica fine particle having a primary particle size of less than 0.1 μm.

10. A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to claim 4, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity, and
3 to 20 parts by mass of the (C2) hydrophobized silica fine particle having a primary particle size of less than 0.1 μm.

11. A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to claim 5, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity, and
3 to 20 parts by mass of the (C2) hydrophobized silica fine particle having a primary particle size of less than 0.1 μm.

12. A kit of a two-step type dental adhesive composition comprising a first agent and a second agent which is the dental curable composition, wherein the kit is used by applying the first agent to an adherend and then further applying the second agent thereto,
wherein the first agent comprises, relative to 100 parts by mass of the first agent,
2 to 30 parts by mass of the (A1) polymerizable monomer having an acidic group,
0 to 50 parts by mass of the (A2) polymerizable monomer having no acidic group,
5 to 50 parts by mass of the (D) water, and
0 to 50 parts by of the (E) volatile organic solvent, and
wherein the dental adhesive composition consists of the second agent and the first agent,
wherein the second agent is the second agent according to claim 6, comprising relative to 100 parts by mass of the (A) polymerizable monomer comprised in the second agent,
0.5 to 5.0 parts by mass of the (B) polymerization initiator,
40 to 120 parts by mass of the (C1) inorganic filler having X-ray radiopacity, and
3 to 20 parts by mass of the (C2) hydrophobized silica fine particle having a primary particle size of less than 0.1 μm.
